(19) United States
(12) Patent Application Publication
(10) Pub. No.: US 2026/0283665 A1
(43) Pub. Date: Sep. 24, 2026

(54) INTERVERTEBRAL DEVICES, AND ASSOCIATED SYSTEMS AND METHODS

(71) Applicant: BLOOM BIOMEDICAL, INC., Irvine, CA (US)

(72) Inventors: Jason Liauw, Laguna Beach, CA (US); Nicholas Hu, Irvine, CA (US); Philippe Marchand, Montreal (CA)

(21) Appl. No.: 19/339,281

(22) Filed: Sep. 24, 2025

Related U.S. Application Data
(60) Provisional application No. 63/699,121, filed on Sep. 25, 2024.

(51) Int. Cl. A61B 17/88; A61B 17/00; A61B 17/56

(52) U.S. Cl. CPC A61B 17/8855; A61B 2017/00261; A61B 2017/564

(57) ABSTRACT

Spinal surgical procedures for implanting intervertebral devices, and associated devices and systems are described herein. A representative spinal surgical procedure can include gaining access to a diseased disc of a spine of a patient via a minimally-invasive a lateral, transpedicular, transfacet, or transforaminal access pathway. Various instruments can be inserted through the trocar to (i) remove some or all of the diseased disc, (ii) expand a disc space around the diseased disc, (iii) insert an intervertebral device into the disc space, and/or (iv) fill the intervertebral device with a fill material. Each of the aforementioned steps can be performed through the minimally-invasive access pathway provided by the trocar. This can minimize disruption to the flesh of the patient—minimizing patient pain and recovery time.

### CROSS-REFERENCE TO RELATED APPLICATION

This application claims priority to U.S. Provisional Application No. 63/699,121, filed Sep. 25, 2024, and titled “INTERVERTEBRAL DEVICES, AND ASSOCIATED SYSTEMS AND METHODS.” This application also includes contains subject matter related to U.S. patent application Ser. No. 18/619,125, filed Mar. 27, 2024, and titled “INTERVERTEBRAL DEVICES, AND ASSOCIATED SYSTEMS AND METHODS,” which is incorporated by reference herein in its entirety.

### TECHNICAL FIELD

The present technology is directed to intervertebral devices, such as intervertebral fusion devices, that can be deployed minimally invasively, and associated system and methods.

### BACKGROUND

Degenerative joint disease in the spine usually involves the tandem degeneration of the intervertebral disc and the two posterior facet joints which act as a tripod to provide stabilization between the vertebrae of the spine. Degeneration of these joints are respectively termed disc degenerative disease (DDD) or disc arthropathy, and facet degenerative disease or facet arthropathy. The result of DDD is often thinning of the disc and a collapse of the disc height. The neural foramen are the openings through which the spinal nerve roots go through when leaving the spinal column, and the height of these foramina directly correspond to the height of the intervertebral disc. When the intervertebral disc height collapses, the natural height of the neural foramina also collapses and, accordingly, the exiting nerve root is compressed which can elicit nerve pain or radicular pain down the legs. Intervertebral disc height collapse can further cause ligamentous laxity and bulging in the spine. These ligaments, namely the posterior longitudinal ligament (PLL) and ligamentum flavum, surround the spinal cord and can bulge into the spinal canal as the disc height collapses. The result is compression of the whole spinal cord and/or the thecal sac positioned centrally in the spinal canal, which can cause radicular pain down the legs in addition to weakness and fatigue in the legs, (e.g., neurogenic claudication).

The surgical treatment of back pain and sciatic pain include etiologies of pain which all stem from disc degenerative joint disease in the spine. Surgical treatments include treating mechanical instability of the spine, treating nerve root compression, and restoring natural alignment of the spine, among others.

Mechanical instability is a result of degeneration of both the intervertebral disc and/or the posterior facet joints. The mechanical instability causes painful arthritic pain or arthropathy. Treating mechanical instability of the spine can include spinal fixation. Much of spinal fixation is geared toward reducing mechanical instability and thereby reducing the movement of arthritic joints that get inflamed with movement. There are numerous methods of spinal fixation including, for example, surgically implanting anterior column and/or posterior column fixation devices. The most pervasive spinal fixation method to this day involves posterior screws used in combination with interbody cages for anterior column support.

As described above, nerve root compression can occur centrally around the spinal cord or thecal sac, or laterally around the exiting nerve root at the neural foramen. Pain from nerve root compression often travels according to a nerve root dermatomal distribution, causing radicular or sciatic pain. Spinal surgical decompression is one method of treating nerve root compression, and aims to remove nerve pain by taking the compression off the nerve roots. This can be accomplished directly through removal of bone/ligament/disc compressing the nerve. It can also be accomplished indirectly by mechanically increasing the intervertebral height with an intervertebral interbody spacer or interlaminar spacer, thereby restoring neural foraminal height and reducing the bulging of the ligaments/disc that bulge into the spinal canal centrally.

One of the newer paradigms in spinal fixation and fusion is restoring natural alignment of the spine. For example, sagittal balance can be restored with intervertebral spacers that restore natural lordotic curvature of the spine. The restoration of neutral spinal alignment enables the patient to walk or stand with good posture rather than leaning forward. This reduces the strain on the paraspinal musculature in the spine. Not restoring natural spinal alignment when performing spinal fixation often results in “flat back syndrome,” in which patients have chronic lower back pain due to muscular fatigue. Additionally, inducing natural sagittal balance with the use of lordotic spacers has become a mainstay in preventing degeneration at the adjacent intervertebral spaces above and below a spinal fusion.

Vertebral interbody spacers have become an important part of spinal fixation for reasons which relate to the fundamentals of treating back pain and nerve pain—for example, treating mechanical instability, treating nerve root compression, and restoring the natural alignment of the spine. Intervertebral spacers can improve the treatment of mechanical instability. For example, the insertion of an intervertebral spacer provides for more anterior support in fixation of the spine. This helps stabilize mechanical instability. Some intervertebral spacers with large footprints can be used as standalone fixation devices. They can also be used in conjunction with posterior spinal fixation, as they add anterior column support allowing for more rigid stabilization which prevents hardware loosening and failure of fusion.

Intervertebral spacers can also improve treatment of nerve root compression. For example, intervertebral spacers can be used to increase the height of a collapsed and degenerated intervertebral disc space. This allows for indirect restoration of the height of the associated neural foramina which can relieve compression of the spinal nerve root exiting at that vertebral level. Additionally, increasing the height of the intervertebral disc space can restore tension to collapsed and bulging ligaments in the spinal canal, namely the PLL and ligamentum flavum. Restoring tension to these ligaments through distraction, termed ligamentum taxis, can reduce the bulging of these ligaments into the spinal canal. The combination of decompression of the neural foramina and spinal canal can reduce radicular sciatic pain and improves neurogenic claudication.

Intervertebral spacers can also help restore the natural alignment of the spine. For example, when the spine falls out of neutral global alignment—which predominantly refers to being hunched forward or out of sagittal alignment—the patient will experience muscular pain as the back strains throughout the day in an attempt to force the patient into a more neutral posture. The lumbar spine has built in natural lordosis which allows us to stand in an upright neutral position. As discs degenerate, thin, and collapse in height, the lumbar spine often loses that lordosis, which is why older patients with degenerated spines are often hunched forward. The popularity of intervertebral spacers has been driven by the fact that restoration of disc height or anterior column height can help bring a patient's spine into a more neutral or lordotic position. In this manner, a harmony of spinal balance can be achieved with a fusion. Indeed, there has been a rise in use of lordotic and hyperlordotic intervertebral spacers which further induce lordosis in the lumbar spine to help compensate for the kyphosis at other degenerated levels. Fusing a spine without the use of intervertebral spacers in the past often resulted in spinal fixation in a flat or kyphotic position which can leave a patient in chronic pain, termed “flat back” syndrome. Fusing without the use of intervertebral spacing is slowly becoming antiquated. There are currently no existing percutaneous interbody systems that allow for specified lordotic correction in the spine.

Interbody fusion implants can be placed into the disc space through either posterior, lateral, or anterior approach trajectories. The two posterior approaches are a posterior lumbar interbody fusion (PLIF) in which an interbody fusion implant is placed through a laminectomy, and transforaminal lumbar interbody fusion (TLIF) in which the facet joint is resected and the interbody fusion implant is placed through a postero-lateral trajectory. A lateral approach to the spine for placement of an interbody fusion implant is termed lateral lumbar or extreme lateral lumbar interbody fusion (LLIF/XLIF). The two anterior approaches to the lumbar spine are a directly anterior open approach, termed anterior lumbar interbody fusion (ALIF) or an antero-lateral approach, termed oblique lumbar interbody fusion (OLIF). Each of these approaches, with the exception of ALIF, can be performed through either an open or minimally invasive approach using retractors. Current minimally-invasive interbody fusion implants utilize an oblique postero-lateral approach with similar trajectory to a TLIF, but necessitate a slightly more lateral trajectory to get under the facet joint, targeting the Kambin's triangle. Typically, the facet is not removed with these approaches but rather the disc space is dilated in the Kambin's triangle.

One drawback with insertion of interbody cages is that there is still a significant amount of dissection and tissue trauma with open or minimally invasive open approaches to gain access to the disc space which equates to more post-operative pain and longer recovery. The use of more minimally invasive retractors allows for less tissue trauma, but still involves tissue retraction and retraction of the nerve root potentially—which can result in non-trivial post-operative pain. Further, the current approach with more minimally invasive retractors is to use smaller interbody implants that fit through the access port. However, a drawback of using smaller implants is that there is less contact surface area with the vertebrae above and below, leading to poor support which increases the risk of subsidence of the implant itself into the adjacent vertebrae. Percutaneous approaches to the spine with tubular dilators are an approach used to even further limit tissue dissection and retraction, but adoption has been limited due to poor visualization of the exiting nerve at the Kambin's triangle, and the risk of nerve injury when trying to dilate within a collapsed foramen where the safe zone of the Kambin's triangle is even narrower. Additionally, current percutaneous techniques are unfamiliar to many surgeons requiring additional training, and the unfamiliarity can often increase procedure time and risk.

Current development in interbody fusion devices and techniques, in addition to minimizing the approach, have also focused on the restoration of disc height and lordosis to restore spinal alignment. Existing open and minimally invasive techniques employ the use of instrumentation such as rasps, curettes, shavers, and dilators to clear the disc space and release the vertebral body ligamentous attachments to enable distraction of the intervertebral space. A lot of these instruments when used in an endoscopic or minimally invasive approach are hindered by the inability of the system instrumentation to enable the surgeon to prepare the disc space adequately for the deployed geometry of the implant since access is constrained during the procedure.

There are a number of existing choices of interbody fusion devices. Original interbody fusion devices were static polyetheretherketone (PEEK) or metal cages. To enable improved lordotic correction, these static cages were either shaped with built in lordotic angulation or were inserted and packed on the anterior most portion of the vertebral body and screws were compressed posteriorly to induce lordosis. The drawback of both static and expandable posteriorly inserted cages, however, remains that they often subside due to their small footprint and/or contact surface area on the vertebral endplate along with the fact that they do not conform to the vertebral body well which leads to point loading and endplate fracturing. Expandable cages especially are more susceptible to causing endplate fractures as expansion causes higher forces at the endplate/implant interface. Expandable cages that focus on inducing lordosis expand along the anterior wall but also cause point loading along the implant. These expandable cages can also reduce the overall contact surface area by lifting the vertebrae away from the more posterior portions of the rigid implant structure.

While lateral and anteriorly placed cages do possess more surface area and thereby have improved endplate coverage, they still engender a separate incision and dissection for the approach. Currently, endoscopically inserted interbody fusion devices can only expand in height.

Lumbar intervertebral fusion devices are indicated for use in skeletally mature patients with DDD at one, two, or more than two contiguous levels from L2-S1. DDD is defined as back pain of discogenic origin with degeneration of the disc confirmed via history and radiographic studies. Patients with DDD can also have spondylolisthesis at the involved level(s). Intervertebral devices are indicated to be used with a supplemental fixation system and autograft bone. Intervertebral fusion devices aim to restore disc height and lumbar lordosis. There are several methods of insertion for these devices, with limitations varying across the different approaches to insertion. Many insertion methods adopt either an anterior or posterior approach, where anterior insertion entails a greater risk for complications, but accomplishes superior restoration of height and lumbar lordosis.

Due to the nature of the incisions and complications involved with anterior methods of inserting intervertebral devices, surgeons are gravitating towards posterior methods of insertion. Specifically, the Transforaminal Lumbar Interbody Fusion (TLIF) approach has come to dominate the field of interbody fusion. However, insertion at a posterior-lateral angle comes with the limitation to the footprint size, which can result in higher incidence of endplate fractures and subsidence, given the small window of insertion limits the size of an interbody.

### BRIEF DESCRIPTION OF THE DRAWINGS

Many aspects of the present technology can be better understood with reference to the following drawings. The components in the drawings are not necessarily to scale. Instead, emphasis is placed on clearly illustrating the principles of the present technology.

FIGS. 1A and 1B are a side view and a top view, respectively, of a portion of a spine illustrating an access step of a spinal surgical procedure in accordance with embodiments of the present technology.

FIGS. 1C and 1D are a side view and a top view, respectively, of a portion of the spine illustrating a discectomy step of the spinal surgical procedure in accordance with embodiments of the present technology.

FIGS. 1E and 1F are side views of a portion of the spine illustrating a first stage and a second stage of a balloon deployment step of the spinal surgical procedure, respectively, in accordance with embodiments of the present technology.

FIGS. 1G and 1H are corresponding top views of the portion of the spine shown in FIGS. 1E and 1F, respectively, illustrating the first stage and the second stage of the balloon deployment step in accordance with embodiments of the present technology.

FIGS. 1I-1K are a side view, another side view, and a top view, respectively, of a portion of the spine illustrating an intervertebral device deployment step of the spinal surgical procedure in accordance with embodiments of the present technology.

FIGS. 1L-1N are a side view, another side view, and a top view, respectively, of a portion of the spine illustrating a first stage of an intervertebral device fill step of the spinal surgical procedure in accordance with embodiments of the present technology.

FIGS. 1O-1Q are a corresponding side view, another side view, and a top view of the portion of the spine shown in FIGS. 1L-1N, respectively, illustrating a second stage of the intervertebral device fill step in accordance with embodiments of the present technology.

FIG. 1R is an enlarged side view of a portion of the spine illustrating an intervertebral device closure step of the spinal surgical procedure in accordance with embodiments of the present technology.

FIG. 1S is a side view of a portion of the spine of the patient illustrating a posterior fixation step of the spinal surgical procedure in accordance with embodiments of the present technology.

FIG. 2 is a flow diagram of a process or method for performing a spinal surgical procedure in accordance with embodiments of the present technology.

FIG. 3A is a side view of a portion of a spine illustrating a first posterior fixation step of a spinal surgical procedure in accordance with embodiments of the present technology.

FIG. 3B is a side view of a portion of the spine illustrating a second posterior fixation step of the spinal surgical procedure in accordance with embodiments of the present technology.

FIG. 3C is a side view of a portion of the spine illustrating a third posterior fixation step of the spinal surgical procedure in accordance with embodiments of the present technology.

FIG. 3D is a side view, including an enlarged portion, of a portion of the spine illustrating an access step of the spinal surgical procedure in accordance with embodiments of the present technology.

FIGS. 3E and 3F are side views, including enlarged portions, of a portion of the spine illustrating distraction steps of the spinal surgical procedure in accordance with embodiments of the present technology.

FIGS. 3G-3I are side views, including enlarged portions, of a portion of the spine illustrating distraction steps of the spinal surgical procedure in accordance with additional embodiments of the present technology.

FIGS. 3J and 3K are side views of a portion of the spine illustrating posterior fixation locking steps of the spinal surgical procedure in accordance with embodiments of the present technology.

FIG. 3L is a side view, including an enlarged portion, of a portion of the spine illustrating an intervertebral device deployment step of the spinal surgical procedure in accordance with embodiments of the present technology.

FIG. 3M is a side view of a portion of the spine illustrating finally-implanted first and second intervertebral devices and a posterior fixation assembly in accordance with embodiments of the present technology.

FIGS. 4A and 4B are side views of a portion of a spine before and after inflation of a balloon in accordance with embodiments of the present technology.

FIG. 5 is a flow diagram of a process or method for performing a spinal surgical procedure in accordance with embodiments of the present technology.

FIGS. 6A and 6B are side views of a portion of a spine illustrating distraction steps (e.g., parallel distraction steps) of a spinal surgical procedure in accordance with embodiments of the present technology.

FIGS. 7A and 7B are side views of the portion of the spine of FIGS. 6A and 6B illustrating distraction steps (e.g., a lordosis and distraction steps) of the spinal surgical procedure in accordance with additional embodiments of the present technology.

FIG. 8A is a perspective view of a portion of a posterior fixation assembly of FIGS. 7A and 7B including a fixation member in accordance with embodiments of the present technology.

FIG. 8B is a partially-transparent perspective view of a tulip of the fixation member of FIG. 8A in accordance with embodiments of the present technology.

FIG. 8C is a partially-transparent perspective view of the tulip and a screw body of the fixation member of FIG. 8A in accordance with embodiments of the present technology.

FIG. 8D is a partially-transparent perspective view of the fixation member and a spanning member of FIGS. 8A-8C in accordance with additional embodiments of the present technology.

FIG. 9 is a perspective view of a differential torque driver in accordance with embodiments of the present technology.

FIG. 10A is a side view of a portion of a spine of a patient illustrating a navigation and trajectory planning step of a spinal surgical procedure in accordance with embodiments of the present technology.

FIG. 10B is a side view of a portion of the spine illustrating an access step of the spinal surgical procedure in accordance with embodiments of the present technology.

FIG. 10C is a side view and an enlarged front view of a portion of the spine illustrating a first intervertebral device deployment step of the spinal surgical procedure in accordance with embodiments of the present technology.

FIG. 10D is a side view and an enlarged front view of a portion of the spine illustrating a second intervertebral device deployment step of the spinal surgical procedure in accordance with embodiments of the present technology.

FIG. 11 is a perspective view of a distal portion of a discectomy device in accordance with embodiments of the present technology.

FIG. 12A is a side view of a balloon device deployed and expanded within a disc space of a spine of a patient in accordance with embodiments of the present technology.

FIG. 12B is a side view of a pressure-relief valve of the balloon device of FIG. 12A coupled to a portion of an inflation shaft of the balloon device in accordance with embodiments of the present technology.

FIGS. 13A-13C are a top view, a back view, and a side view, respectively, of an intervertebral device deployed and expanded within a disc space of a spine of a patient in accordance with embodiments of the present technology.

FIG. 14A is a perspective view of an intervertebral device in accordance with embodiments of the present technology.

FIG. 14B is an enlarged perspective view of a portion of the intervertebral device of FIG. 14A in accordance with embodiments of the present technology.

FIG. 14C is a graph illustrating a height of the intervertebral device of FIG. 14A in millimeters along the Y-axis versus a loading force in pounds along the X-axis in accordance with embodiments of the present technology.

FIG. 15A illustrates several suture anchors in accordance with embodiments of the present technology.

FIG. 15B illustrates another suture anchor secured within bone of a patient in accordance with embodiments of the present technology.

FIG. 15C is a top view of a trocar for providing access to a vertebra or disc of a spine of a patient including one of the suture anchors of FIGS. 15A and/or 15B in accordance with embodiments of the present technology.

FIG. 16 is a perspective view of a trocar for providing access to a vertebra or disc of a patient in accordance with additional embodiments of the present technology.

### DETAILED DESCRIPTION

Aspects of the present technology are directed generally toward intervertebral devices, such as intervertebral fusion devices, that can be deployed minimally invasively or percutaneously, and associated system and methods. In several of the embodiments described below, a method for performing a spinal surgical procedure to implant an intervertebral device can include inserting a trocar into a patient to proximate to a diseased disc via a lateral, transpedicular, transfacet, transforaminal, trans-iliac, trans-sacral, and/or other approach. The trocar can provide an access pathway for subsequent instruments to be inserted thereto for treating the diseased disc. The method can further include inserting a discectomy device through the trocar and using the discectomy device to remove some or all of the diseased disc to form a disc space. Next, a balloon can be inserted through the trocar into the disc space and expanded to further disrupt and/or clear any remaining portion of the diseased disc and to lift an upper vertebra adjacent the diseased disc relative to a lower vertebra adjacent the diseased disc (e.g., to create lordosis). Then, the intervertebral device can be inserted through the trocar into the disc space and expanded within the disc space. The intervertebral device can comprise a braid, weave, mesh, and/or the like of filaments. Next, the intervertebral device can be filled with a fill material, such as a plurality of particles that form a gabion-like structure when loaded. In some embodiments, the intervertebral device is tensioned to better pack the fill material therein and/or to induce the gabion-like structure. The intervertebral device can then be closed to inhibit or even prevent egress of the fill material and released (e.g., from a delivery shaft) within the disc space. Finally, a posterior fixation assembly can be attached to the upper and lower vertebrae adjacent the disc space to stabilize the vertebrae and provide for bone ingrowth into the intervertebral device and the fill material therein.

Notably, each of the steps of the spinal surgical method can be performed through the open surgery, minimally-invasive, or percutaneous port/access pathway provided by the trocar. In some aspects of the present technology, this can minimize disruption to the tissue of the patient—minimizing patient pain and recovery time. Furthermore, the spinal surgical method can traverse safer pathways that require smaller size compared to conventional techniques. For example, the trocar and various instruments can be sized to fit through a transpedicular, trans-iliac, trans-sacral, and/or other approach that encompasses a pathway (e.g., corridor) of less than 4.5 millimeters.

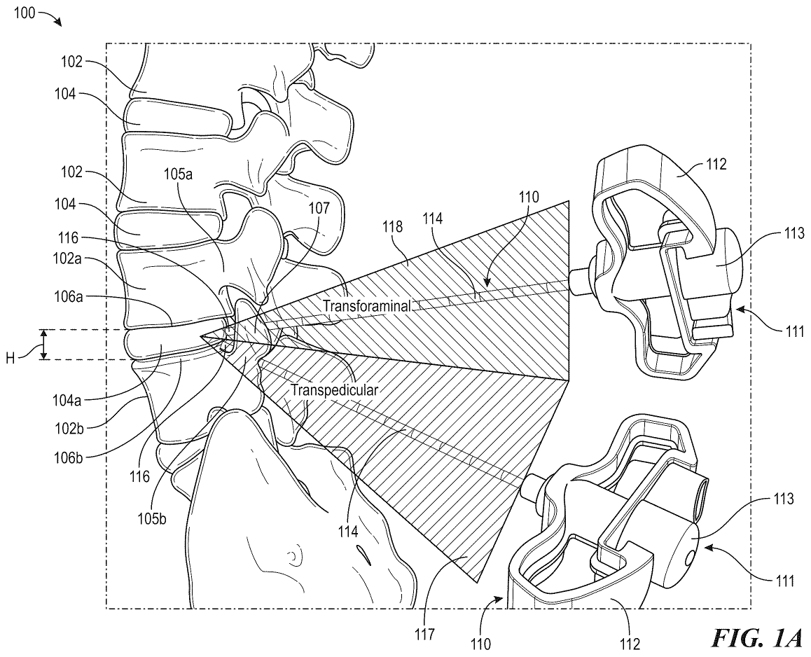

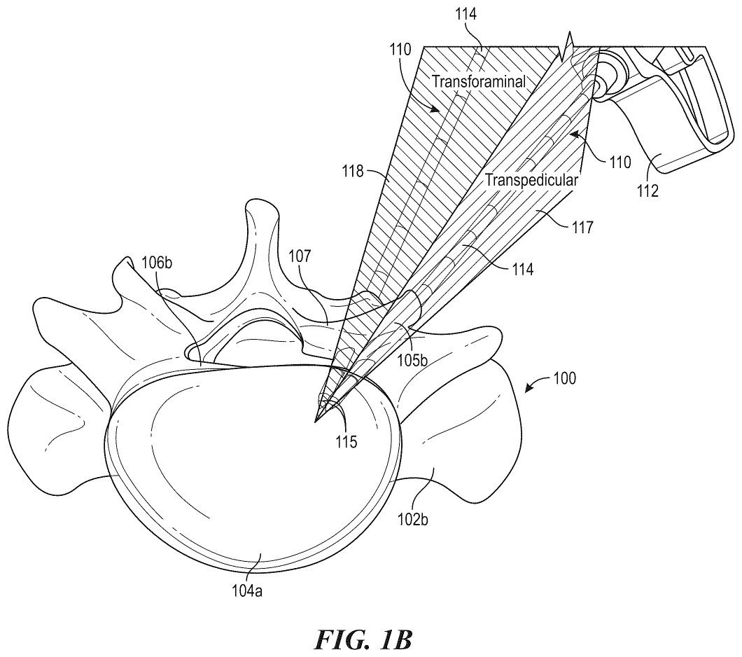

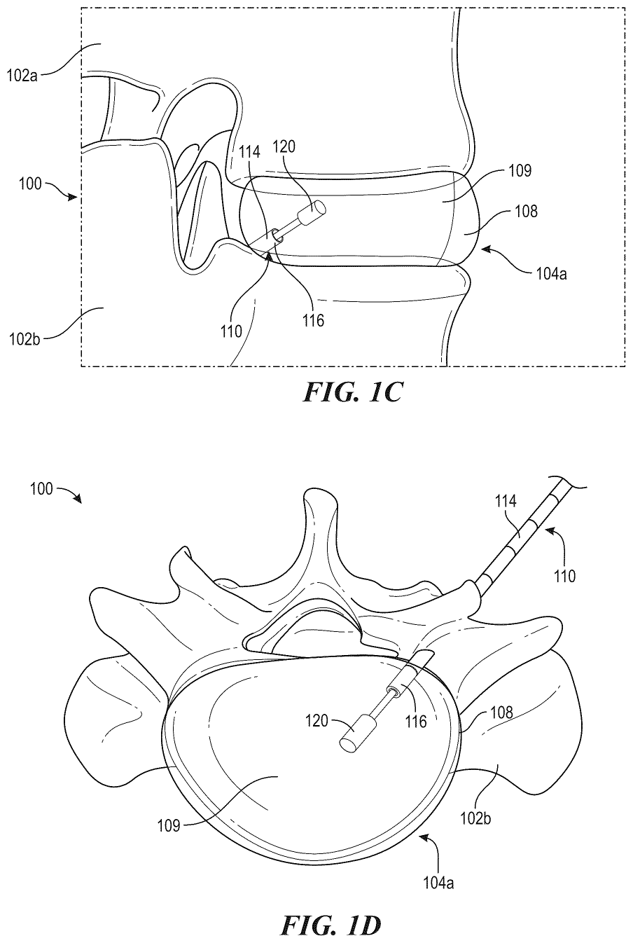

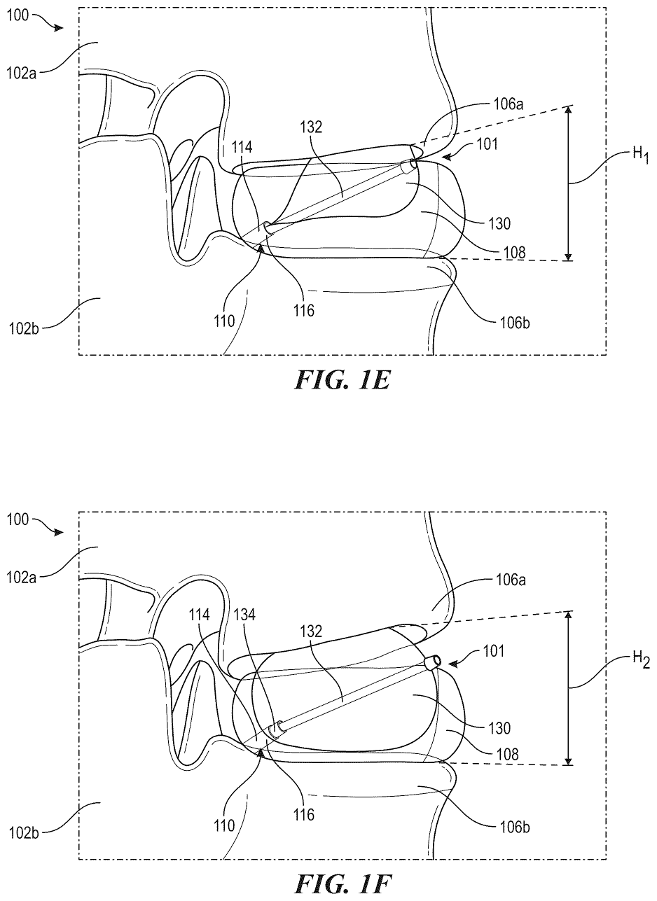

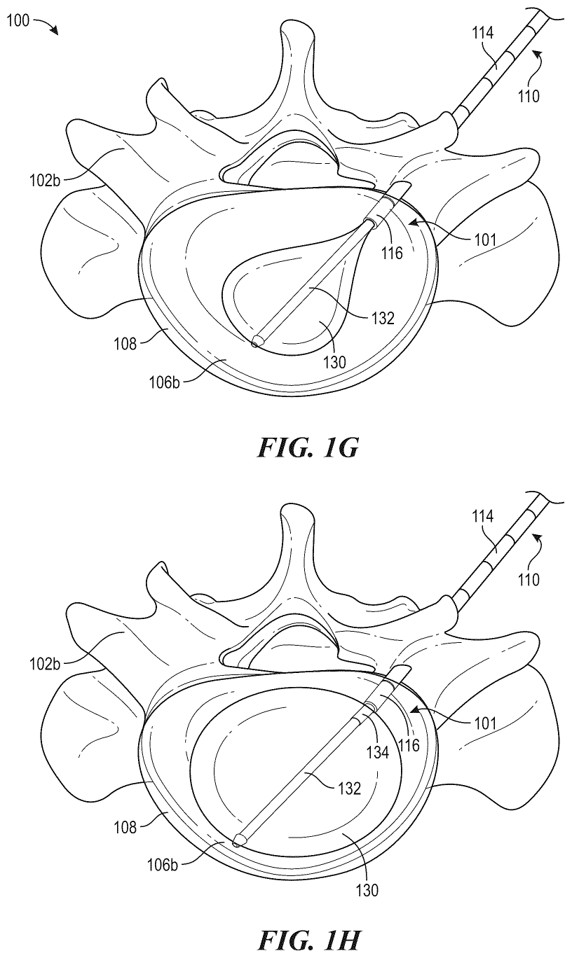

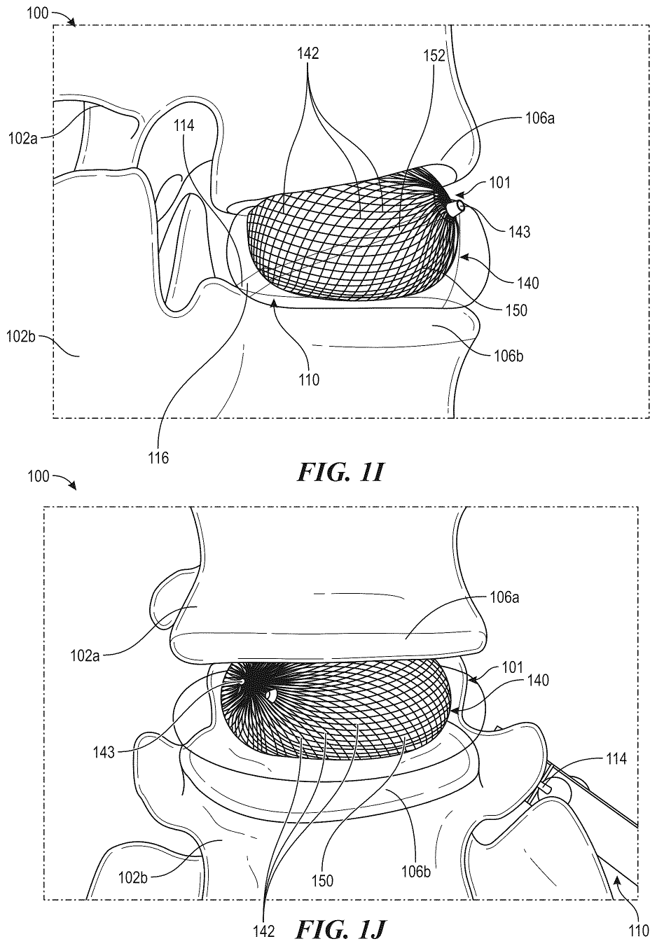

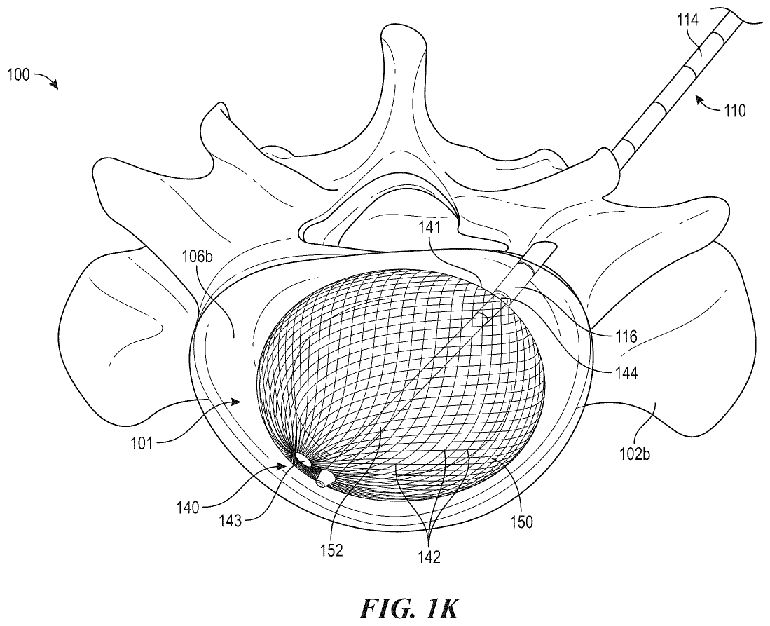

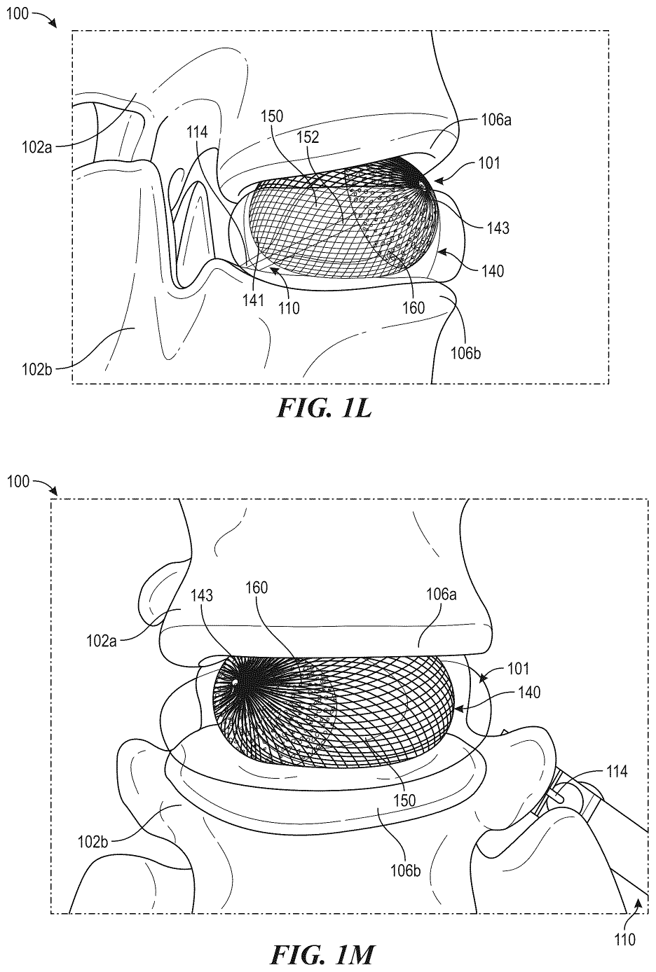

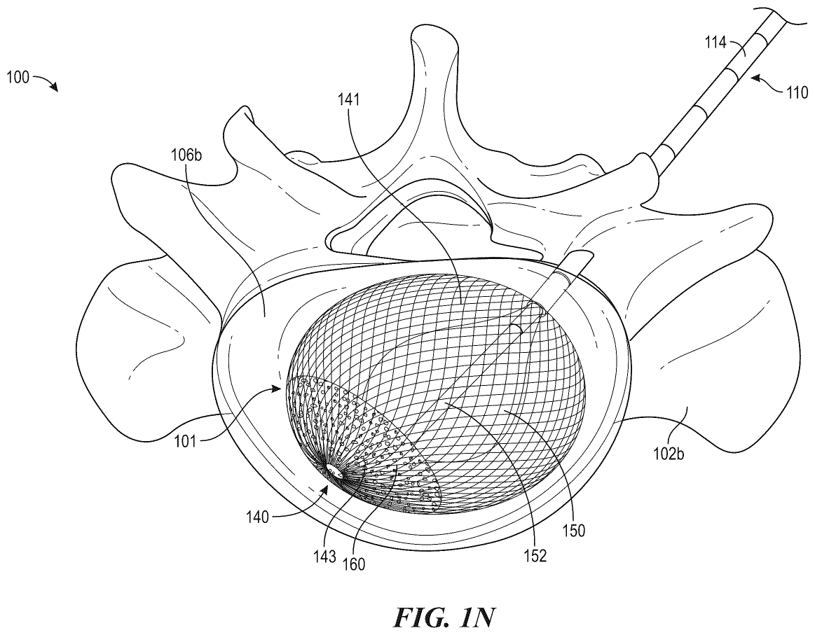

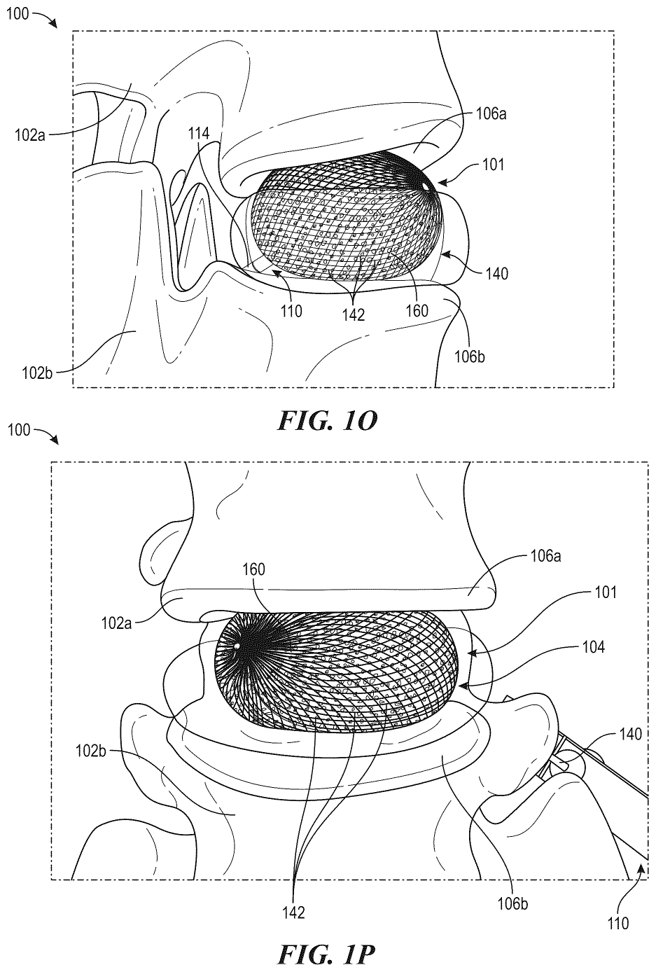

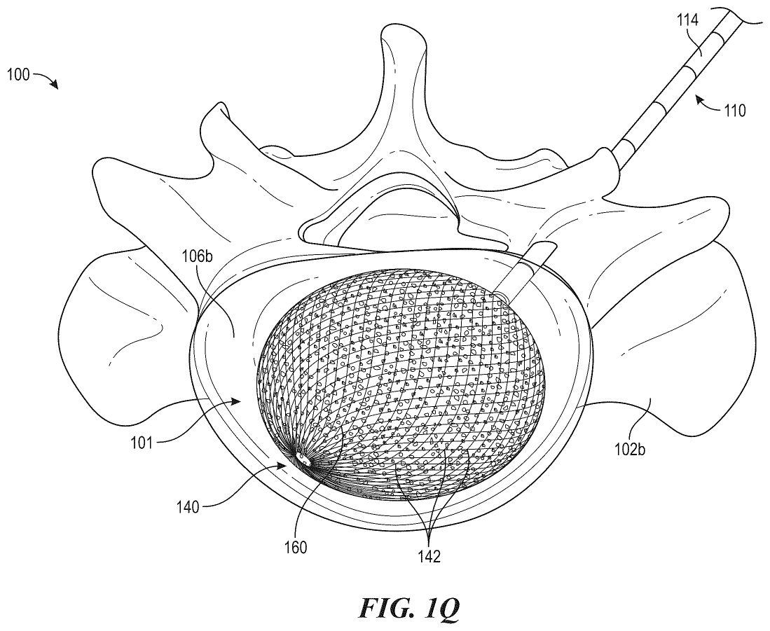

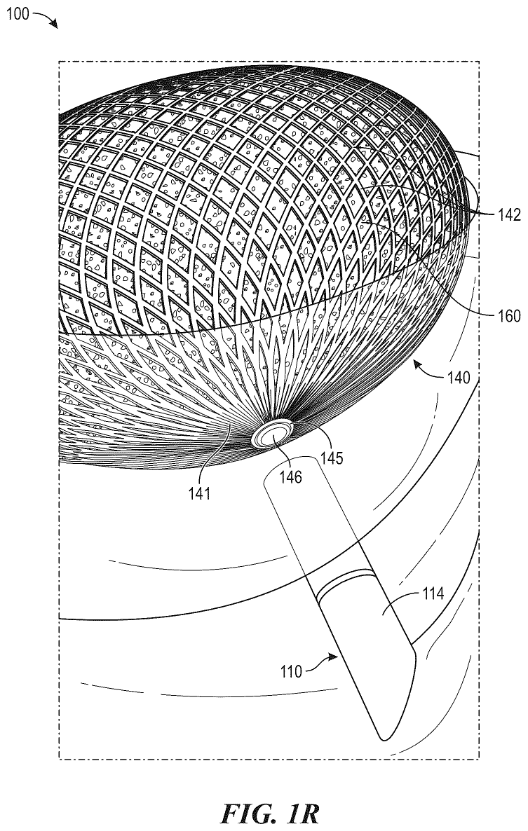

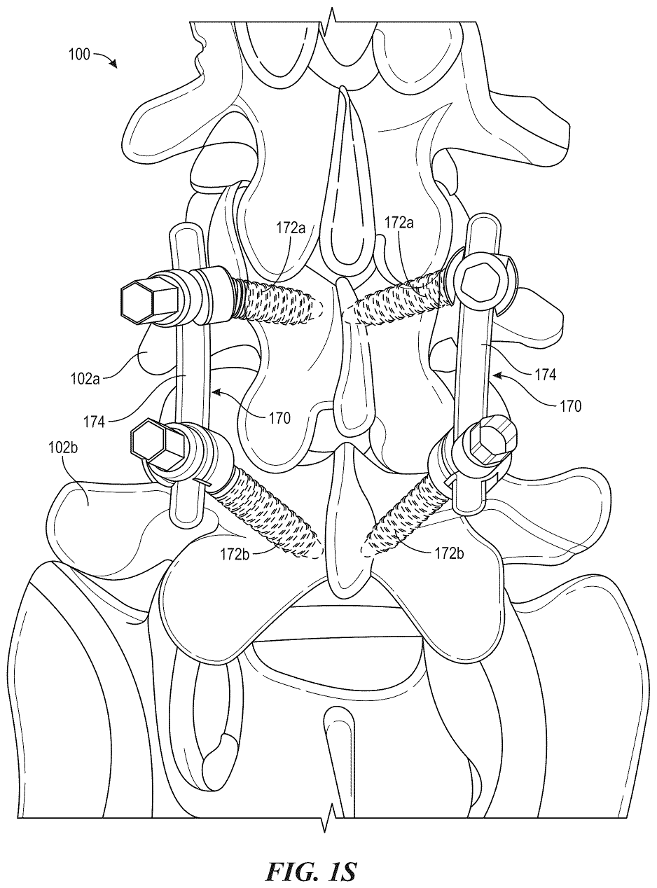

Certain details are set forth in the following description and in FIGS. 1A-16 to provide a thorough understanding of various embodiments of the present technology. In other instances, well-known structures, materials, operations, and/or systems often associated with spinal surgical procedures, intervertebral devices, spinal fusion procedures, posterior fixation assemblies, suture anchors, balloon devices, and the like, are not shown or described in detail in the following disclosure to avoid unnecessarily obscuring the description of the various embodiments of the technology. Those of ordinary skill in the art will recognize, however, that the present technology can be practiced without one or more of the details set forth herein, and/or with other structures, methods, components, and so forth. The terminology used below is to be interpreted in its broadest reasonable manner, even though it is being used in conjunction with a detailed description of certain examples of embodiments of the technology.

With regard to the terms “distal” and “proximal” within this description, unless otherwise specified, the terms can reference a relative position of the portions of a spinal access system with reference to an operator and/or a location in the spinal anatomy. Also, as used herein, the designations “rearward,” “forward,” “upward,” “downward,” and the like are not meant to limit the referenced component to a specific orientation. It will be appreciated that such designations refer to the orientation of the referenced component as illustrated in the Figures; the systems of the present technology can be used in any orientation suitable to the user. Moreover, the terms “distal” and “proximal” can additionally be referred to as “leading” and “trailing,” respectively, and/or the like.

The accompanying Figures depict embodiments of the present technology and are not intended to limit its scope unless expressly indicated. The sizes of various depicted elements are not necessarily drawn to scale, and these various elements may be enlarged to improve legibility. Component details may be abstracted in the Figures to exclude details such as position of components and certain precise connections between such components when such details are unnecessary for a complete understanding of how to make and use the present technology. Many of the details, dimensions, angles and other features shown in the Figures are merely illustrative of particular embodiments of the disclosure. Accordingly, other embodiments can have other details, dimensions, angles and features without departing from the present technology. In addition, those of ordinary skill in the art will appreciate that further embodiments of the present technology can be practiced without several of the details described below.

To the extent any materials incorporated herein by reference conflict with the present disclosure, the present disclosure controls. The headings provided herein are for convenience only and should not be construed as limiting the subject matter disclosed.

### I. Selected Embodiments of Intervertebral Devices, Systems, and Methods

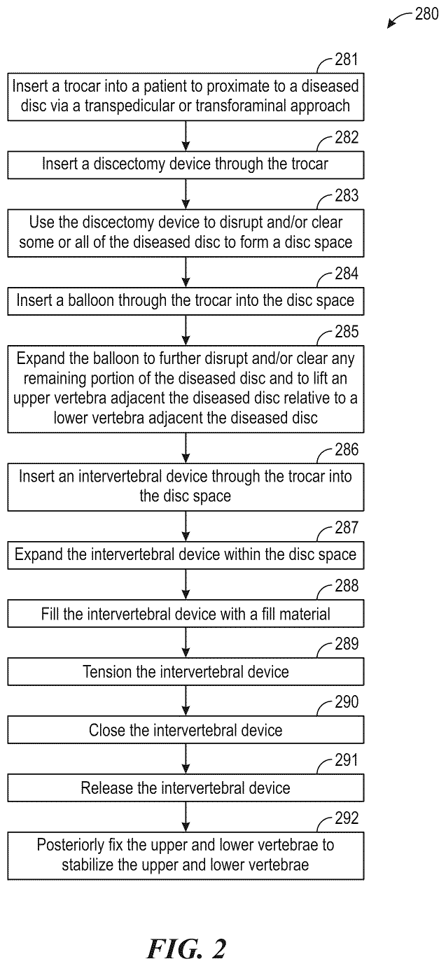

FIGS. 1A-1S are different views of a spinal surgical procedure (e.g., a spinal surgical method) on a spine 100 of a patient in accordance with embodiments of the present technology. The spinal surgical procedure can be a spinal fusion procedure (e.g., a single-level fusion) in which an existing diseased disc of the spine is fully or partially removed, and an intervertebral device is inserted into the disc space to support the adjacent vertebrae and provide for bone ingrowth therein. FIGS. 1A-1S provide an overview of some general aspects/steps of the spinal surgical procedure, and FIGS. 2-16 illustrate additional embodiments and/or aspects of the various steps, devices, and/or systems that can be used therein. In some embodiments, some of the steps of the spinal surgical procedure and/or the devices and systems used therein illustrated in FIGS. 1A-1S can include some features that are at least generally similar in structure and function, or identical in structure and function, to the corresponding features of the devices, systems, and/or methods described in International Patent Application No. PCT/US2021/051829, titled “INTERVERTEBRAL FUSION DEVICE WITH BONE GRAFT LUMBAR,” and filed Sep. 23, 2021, which is incorporated herein by reference in its entirety.

FIGS. 1A and 1B are a side view (e.g., a lateral view) and a top view (e.g., an axial view), respectively, of a portion of the spine 100 illustrating an access step of the spinal surgical procedure in accordance with embodiments of the present technology. Referring to FIGS. 1A and 1B, the spine includes a plurality of vertebrae 102 (including an individually identified first or upper vertebra 102a and a second or lower vertebra 102b) separated by discs 104 (e.g., intervertebral discs; including an individually identified diseased disc 104a). The diseased disc 104a can result from degenerative joint disease and/or disc arthropathy. The result of the diseased disc 104a often is a thinning of the diseased disc 104a and a collapse of a disc height H (FIG. 1A) of the diseased disc 104a. Such collapse of the disc height H can lead to collapse of neural foramina and, accordingly, compression of the exiting nerve root which can elicit nerve pain and/or radicular pain down the legs of the patient. Likewise, collapse of the disc height H can further cause ligamentous laxity and bulging in the spine. The result is compression of the whole spinal cord and/or the thecal sac positioned centrally in the spinal canal, which can cause radicular pain down the legs in addition to weakness and fatigue in the legs (e.g., neurogenic claudication).

In the illustrated embodiment, a trocar 110 is used to access the diseased disc 104a via either a transpedicular or transforaminal (e.g., transfacet) approach. Referring to FIG. 1A, the trocar 110 can include a handle 112 coupled to a hollow cannula 114 defining a lumen. The trocar 110 and/or the cannula 114 can be referred to as a sheath, a shaft, a stylet, an access port, an introducer, a tube, and/or the like. In some embodiments, the handle 112 includes a seal that selectively provides access to the lumen of the cannula 114. In some embodiments during the illustrated access step of the spinal surgical procedure, an introducer 111 is positioned within the cannula 114. For example, the introducer 111 can include a handle 113 (e.g., a proximal portion) that can be selectively locked to the handle 112 of the trocar 110 and an elongate member (obscured by the cannula 114 in FIGS. 1A and 1B; e.g., a needle, an awl) having a tip 115 (FIG. 1B) configured to extend distally out of the cannula 114 past a distal end portion 116 of the cannula 114 when the handles 112, 113 are locked together. The tip 115 can be sharpened, pointed, angled, and/or the like to facilitate insertion of the trocar 110 through the flesh and bone (e.g., the upper vertebra 102a or the lower vertebra 102b) of the patient to proximate to the diseased disc 104a (e.g., near the diseased disc 104a, within the diseased disc 104a). The trocar 110 and the introducer 111 can be pushed, rotated, and/or otherwise advanced through the flesh and bone to proximate the diseased disc 104a.

Referring to FIGS. 1A and 1B, in the illustrated embodiment the transpedicular approach can include advancing the trocar 110 and the introducer 111 through a pedicle 105b and an upper endplate 106b of the lower vertebra 102b. In some embodiments, such an approach can traverse the same insertion trajectory as a subsequent pedicle screw used for posterior fixation (e.g., as described in detail below with reference to FIG. 1S). The transforaminal approach can include advancing the trocar 110 and the introducer 111 through a facet joint 107 between the upper and lower vertebrae 102a-b. Referring to FIG. 1A, in other embodiments the transpedicular approach can include advancing the trocar 110 and the introducer 111 through a pedicle 105a and a lower endplate 106a of the upper vertebra 102a. Both the transpedicular approach, transfacet approach and the transforaminal approach access the diseased disc 104a through bone of the upper and/or lower vertebrae 102a-b along a trajectory that avoids the spinal nerve roots, and therefore avoids retraction of the spinal nerve roots during the spinal surgical procedure which could pose a risk to the patient. Accordingly, in some aspects of the present technology both trajectories can inhibit or even prevent the trocar 110 from contacting and potentially damaging the nerve roots as they can go through bone and circumvent traditional access corridors that go through spaces that the nerves can also be found in. In contrast, many conventional access techniques access the disc space by squeezing around the bony elements of the vertebrae—for example, through openings in which the spinal nerve roots exit the spinal column—increasing the likelihood of nerve damage during access.

Referring to FIGS. 1A and 1B, the transpedicular approach can comprise a variety of different access angles shown as a shaded range 117 and the transforaminal and transfacet approaches can likewise comprise a variety of different access angles shown as a shaded range 118. The specific access angle(s) and trajectory for the trocar 110 can be determined via radiographic (e.g., X-Ray) imaging and/or other medical imaging procedures performed before (e.g., preoperatively) and/or during the spinal surgical procedure (e.g., intraoperatively).

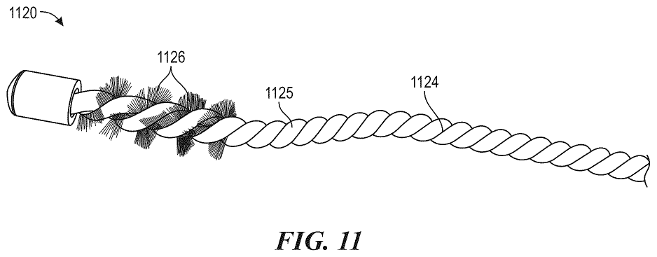

FIGS. 1C and 1D are a side view (e.g., a lateral view) and a top view (e.g., an axial view), respectively, of a portion of the spine 100 illustrating a discectomy step of the spinal surgical procedure in accordance with embodiments of the present technology. Referring to FIGS. 1C and 1D, after accessing the diseased disc 104a, the introducer 111 (FIGS. 1A and 1B) can be removed from within the cannula 114 of the trocar 110 and a discectomy device 120 can be inserted through the cannula 114 past the distal end portion 116 thereof and into the diseased disc 104a. In some embodiments, the discectomy device 120 is self-expanding, bladed, sharpened, rotatable, translatable, and/or otherwise configured to engage, disrupt, and/or dislodge the diseased disc 104a. Further embodiments of discectomy devices are described in detail below with reference to FIG. 11.

The diseased disc 104a can include a ligamentous ring 108 (e.g., comprising an annulus fibrosus, a posterior longitudinal ligament, and/or an anterior longitudinal ligament) surrounding a nucleus 109 (e.g., a nucleus pulposus). The ligamentous ring 108 is shown as partially transparent and the nucleus 109 is shown as transparent in FIGS. 1C and 1D for clarity. The ligamentous ring 108 can connect the upper and lower vertebrae 102a-b and keep the nucleus 109 intact when forces are applied to the spine 100, while the nucleus 109 can provide cushioning between the upper and lower vertebrae 102a-b. Accordingly, the nucleus 109 can be softer and easier to disrupt and remove than the ligamentous ring 108. The discectomy device 120 can be manipulated to engage either or both of the ligamentous ring 108 and the nucleus 109 to clear and remove such material.

In some embodiments, the discectomy device 120 can be inserted through a separate inner trocar (not shown) inserted through the trocar 110, which functions as an outer trocar. The inner trocar can be curved or otherwise shaped to facilitate deployment of the discectomy device 120 to different portions of the diseased disc 104a.

After a sufficient amount of the diseased disc 104a has been removed and/or disrupted, the spinal surgical procedure can include deploying a balloon into the disc space. For example, FIGS. 1E and 1F are side views (e.g., lateral views) of a portion of the spine 100 illustrating a first stage and a second stage of a balloon deployment step of the spinal surgical procedure, respectively, in accordance with embodiments of the present technology. Likewise, FIGS. 1G and 1H are corresponding top views (e.g., axial views) of the portion of the spine 100 shown in FIGS. 1E and 1F, respectively, illustrating the first stage and the second stage of the balloon deployment step in accordance with embodiments of the present technology. Referring to FIGS. 1E-1H together, after a sufficient amount of the diseased disc 104a (FIGS. 1A-1D) has been removed by the discectomy device 120 (FIGS. 1C and 1D), the discectomy device 120 can be removed from the cannula 114 of the trocar 110 and a first balloon 130 can be inserted through the cannula 114 past the distal end portion 116 into a disc space 101 (e.g., within any remaining portion of the ligamentous ring 108) where the diseased disc 104a (FIGS. 1A-1D) has been fully or partially removed in the discectomy step. The first balloon 130 can have a distal portion coupled to an inner balloon shaft 132 and a proximal portion coupled to an outer balloon shaft 134 (obscured in FIGS. 1E and 1G) that are advanceable through the cannula 114. The first balloon 130, the inner balloon shaft 132, and the outer balloon shaft 134 can be collectively referred to as a balloon device or a balloon expansion device.

In the first stage shown in FIGS. 1E and 1G, the first balloon 130 is partially inserted into the disc space 101 and partially inflated within the disc space 101. The first balloon 130 can be inflated via an external pressure source coupled to a lumen of the outer balloon shaft 134. In the second stage shown in FIGS. 1F and 1H, the first balloon 130 is fully inserted into the disc space 101 and fully inflated within the disc space 101.

Expansion of the first balloon 130 within the disc space 101 can act to break, distract, and/or otherwise disrupt any remaining portion of the diseased disc 104a, such as some or all of the ligamentous ring 108. More specifically, the first balloon 130 can expand horizontally (e.g., along a plane extending between the lower and upper endplates 106a-b) to directly disrupt and break the ligamentous ring 108 and/or can expand vertically to indirectly disrupt and break the ligamentous ring 108 by moving the upper and lower vertebrae 102a-b away from one another. Likewise, as best seen in FIGS. 1E and 1F, inflating the first balloon 130 can enlarge the disc space 101 by increasing a height of the disc space 101 (e.g., from a first value H₁ shown in FIG. 1E to a second value H₂ shown in FIG. 1F greater than the first value). That is, the first balloon 130 can lift the upper vertebra 102a away from the lower vertebra 102b. The first balloon 130 can be configured (shaped, sized, constructed) to expand to a selected shape to, for example, provide differential lifting of the upper and lower vertebrae 102a-b. In some embodiments, the first balloon 130 expands to contact a substantial portion of the upper endplate 106b of the lower vertebra 102b and/or a substantial portion of the lower endplate 106a of the upper vertebra 102a. In some aspects of the present technology, this can avoid point loading on the lower and upper endplates 106a-b to inhibit or even prevent fracture of the lower and upper endplates 106a-b. In contrast, many conventional techniques expand a disc space via an interbody with significant risk of endplate fracture.

In some embodiments, the first balloon 130 can be inserted through a separate inner trocar (not shown) inserted through the trocar 110, which functions as an outer trocar. The inner trocar can be curved or otherwise shaped to facilitate deployment of the first balloon 130 to a specified portion of the disc space 101. Additionally, a pressure within and/or volume of the first balloon 130 can be monitored to provide feedback to a user (e.g., a surgeon) about a state of breakage of the ligamentous ring 108 and/or a state of lifting of the upper and lower vertebrae 102a-b.

After expanding the first balloon 130 within the disc space 101 to disrupt any remaining portion of the diseased disc 104a and to lift the upper vertebra 102a relative to the lower vertebra 102b, the spinal surgical procedure can include deploying an intervertebral device into the disc space 101. For example, FIGS. 1I-1K are side view (e.g., a lateral view), another side view (e.g., an anteriorly-facing view), and a top view (e.g., an axial view), respectively, of a portion of the spine 100 illustrating an intervertebral device deployment step of the spinal surgical procedure in accordance with embodiments of the present technology. Referring to FIGS. 1I-1K together, after expanding the first balloon 130 (FIGS. 1G and 1H) within the disc space 101, the first balloon 130 can be removed from the cannula 114 of the trocar 110 and an intervertebral device 140 can be inserted through the cannula 114 past the distal end portion 116 into the disc space 101 and expanded within the disc space 101.

The intervertebral device 140 can be referred to as an interbody, an implant, a disc-replacement device, a cage, and/or the like. The intervertebral device 140 can be a braid, mesh, or knit of filaments 142 that terminate and/or are joined together at a proximal portion 141 (e.g., a proximal hub; obscured in FIGS. 1I and 1L) and a distal portion 143 (e.g., a distal hub). The intervertebral device 140 can be coupled to a deployment shaft 144 (obscured in FIGS. 1I and 1J) for advancing the intervertebral device 140 through the cannula 114 and into the disc space 101. For example, the proximal portion 141 of the intervertebral device 140 can be releasably coupled to a distal portion of the deployment shaft 144 such that the intervertebral device 140 can be detached from the deployment shaft 144 after it is suitably positioned within the disc space 101.

In the illustrated embodiment, the intervertebral device 140 has been deployed into the disc space 101 and subsequently expanded within the disc space 101 by a second balloon 150 positioned within the intervertebral device 140. The second balloon 150 can be similar to the first balloon described in detail with reference to FIGS. 1E-1H. For example, the second balloon 150 can be coupled to one or more balloon shafts 152 that are insertable through the cannula 114 (e.g., via a lumen in the deployment shaft 144 coupled to the intervertebral device 140) and that have one or more inflation lumens for inflating the second balloon 150 via an external pressure source. Inflating the second balloon 150 expands the intervertebral device 140 into contact with the lower and upper endplates 106a-b of the upper and lower vertebrae 102a-b, respectively, and can also act to re-enlarge (e.g., re-lift) the disc space 101 by increasing the height of the disc space 101 (e.g., from the first value H₁ shown in FIG. 1E to the second value H₂ shown in FIG. 1F greater than the first value). In some aspects of the present technology, re-enlarging the disc space 101 can be easier—requiring less force—than initially enlarging the disc space 101 with the first balloon 130.

In some embodiments, the second balloon 150 can be made thinner than the first balloon 130 because (i) it is configured to be expanded within the intervertebral device 140 such that it does not contact the lower or upper endplates 106a-b and/or (ii) it does not need to exert as much force as the first balloon 130 to lift the upper vertebra 102a relative to the lower vertebra 102b after previously lifting the upper vertebra 102a with the first balloon 130 and disrupting the ligamentous ring 108 (FIGS. 1G and 1H). Moreover, the second balloon 150 can be thinner and/or smaller than the first balloon 130 while also imparting more force against the upper and lower vertebrae 102a-b when expanded because the intervertebral device 140 acts as a reinforcement for the second balloon 150 that imparts a greater burst resistance to the second balloon 150. In some embodiments, the first balloon 130 and the second balloon 150 can be the same balloon. For example, the first balloon 130 can be initially inserted through the trocar 110 and inflated as shown in the balloon deployment step illustrated in FIGS. 1E-1H, before being deflated and removed from the trocar 110 and subsequently inserted through the deployment shaft 144 of the intervertebral device 140 for expanding the intervertebral device 140.

In some embodiments, the intervertebral device 140 can be configured (e.g., shaped, sized) to maximize a contact surface area between and promote conformance between the upper and/or lower endplates 106a-b and the intervertebral device 140. That is, the intervertebral device 140 can conform to the upper and/or lower endplates 106a-b. Likewise, the intervertebral device 140 can be configured to expand selectively or differentially to lift a certain portion of the upper vertebra 102a more than another portion to restore a natural alignment of the spine 100.

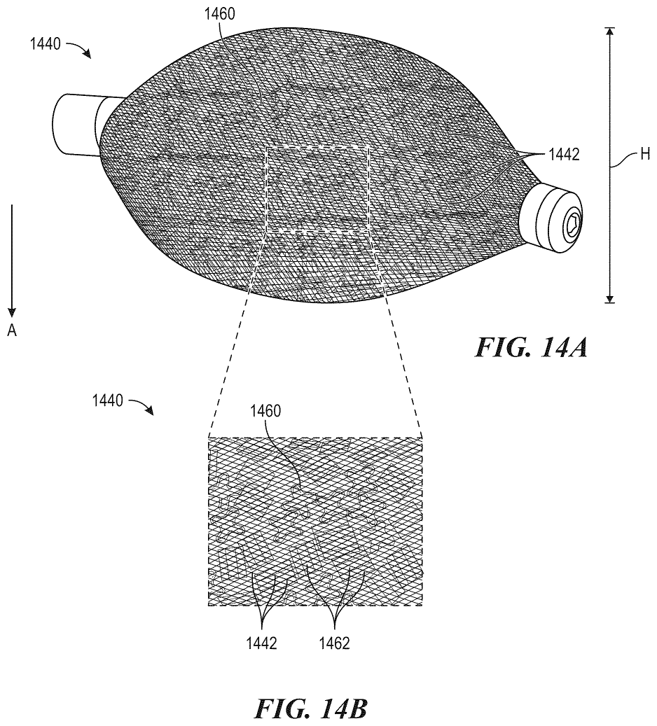

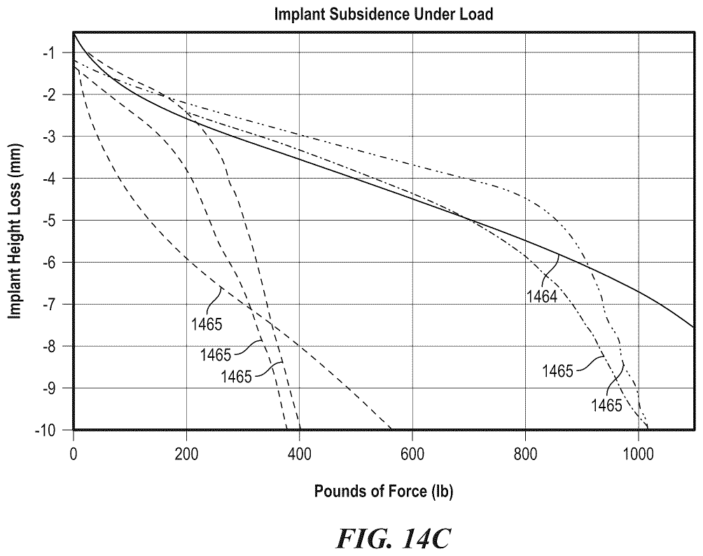

After deploying the intervertebral device 140 in the disc space 101, the spinal surgical procedure can include filling the intervertebral device 140 with a fill material. For example, FIGS. 1L-1N are side view (e.g., a lateral view), another side view (e.g., an anteriorly-facing view), and a top view (e.g., an axial view), respectively, of a portion of the spine 100 illustrating a first stage of an intervertebral device fill step of the spinal surgical procedure in accordance with embodiments of the present technology. Likewise, FIGS. 1O-1Q are a corresponding side view, other side view, and a top view of the portion of the spine 100 shown in FIGS. 1L-1N, respectively, illustrating a second stage of the intervertebral device fill step in accordance with embodiments of the present technology. Referring to FIGS. 1L-1Q, after deploying the intervertebral device 140 and expanding the second balloon 150, the intervertebral device 140 can be filled with a fill material 160. The fill material 160 can comprise a load-bearing material configured to support the upper and lower vertebrae 102a-b in a desired (e.g., lifted) position that restores the height of the disc space 101, and can be configured to promote bone ingrowth therein. As described in greater detail below with reference to FIGS. 14A-14C, the fill material 160 can comprise one or more of a liquid, a gas, and a solid, such as one or more of cement, demineralized bone putty, epoxy, rigid particles, small metal particles, demineralized bone, sand-like particles such as biomaterials, silica particles, ceramic particles, metal particles, bone particles, beads, gabion structures, bone fragments, and/or the like.

Referring to FIGS. 1L-1N, the fill material 160 can be injected into the interior of the intervertebral device 140 through the balloon shaft 152. Accordingly, the second balloon 150 can be partially deflated at the same time the fill material 160 is injected to provide space for the fill material 160. In some aspects of the present technology, the second balloon 150 can at least partially hold the intervertebral device 140 open during filling with the fill material 160 to reduce a resistance of the intervertebral device 140 to filling and/or to guide the fill material 160 to assume a particular geometry within the intervertebral device 140 (e.g., a geometry selected to induce lordosis, kyphosis, and/or the like of the spine 100). In the illustrated embodiment, the distal portion 143 of the intervertebral device 140 is filled with the fill material 160 before the proximal portion 141 (e.g., the intervertebral device 140 is filled in a direction from the distal portion 143 toward the proximal portion 141). In other embodiments, the fill material 160 can be filled in a direction from the proximal portion 141 toward the distal portion and/or in another direction.

In other embodiments, the second balloon 150 and balloon shaft 152 can be removed from the trocar 110 prior to filling the intervertebral device 140 with the fill material 160, and the fill material 160 can be injected directly through the deployment shaft 144 (FIG. 1K) and/or another shaft inserted therethrough. In yet other embodiments, the fill material 160 can be injected into the second balloon 150 such that the fill material 160 fills the second balloon 150. In such embodiments, the second balloon 150 can remain within the intervertebral device 140 after implantation, can be removed (e.g., popped) after filling, or can be made of dissolvable or bioresorbable material.

Referring to FIGS. 1O-1Q, the fill material 160 can entirely or substantially fill the intervertebral device 140 in the second stage of the intervertebral device fill step. Filling the intervertebral device 140 with the fill material 160 can expand (e.g., again expand or partially expand) the intervertebral device 140 into contact with the lower and upper endplates 106a-b of the upper and lower vertebrae 102a-b, respectively, and can also act to re-enlarge (e.g., re-lift) the disc space 101 by increasing the height of the disc space 101 (e.g., from the first value H₁ shown in FIG. 1E to the second value H₂ shown in FIG. 1F greater than the first value). In some embodiments, the intervertebral device 140 can be configured (e.g., shaped, sized) to maximize a contact surface area between and promote conformance between the upper and/or lower endplates 106a-b and the intervertebral device 140 when the intervertebral device 140 is filled with the fill material 160. Likewise, the intervertebral device 140 can be configured to expand selectively or differentially when filled with the fill material 160 to selectively increase a distance between a portion of the upper and lower vertebrae 102a-b (e.g., to lift a certain portion of the upper vertebra 102a) more than another portion to restore a natural alignment of the spine 100. After filling the intervertebral device 140 with the fill material 160, the second balloon 150 can be removed from the cannula 114 of the trocar 110.

In some embodiments, after filling the intervertebral device 140 with the fill material 160, the intervertebral device 140 can be tensioned to, for example, reduce a volume and/or surface area of the intervertebral device 140 to pack the fill material 160 together and/or increase the rigidity of the fill material 160 or overall device. Tensioning the intervertebral device 140 can include exerting a force on the filaments 142 to draw the filaments closer together and/or more tightly together. In some embodiments, tensioning the intervertebral device 140 can happen before or concurrently with filling the intervertebral device 140 with the fill material 160 and/or closing the proximal portion 141 of the intervertebral device 140, as described in detail below with reference to FIG. 1R.

In some embodiments, the intervertebral device 140 is tensioned by filling the intervertebral device 140 with the fill material 160. For example, filling the intervertebral device 140 with the fill material 160 can expand the volume of the intervertebral device 140 against the constraint of the upper and lower vertebrae 102a-b (e.g., a constrained surface area which the intervertebral device 140 contacts)—thereby tensioning the intervertebral device 140. In some embodiments, the intervertebral device 140 is filled to have a generally spherical shape. Such a spherical shape can have the best efficiency of surface area to volume. After the source of pressure used to inject the fill material 160 is removed, or when any balloons (e.g., the second balloon 150) holding the space are removed, the anatomy may exert a compacting/deforming force on the filled intervertebral device 140, flattening it to a less ideal shape. This can create a larger surface area given a consistent volume and thus apply tension to the filaments 142.

After tensioning and filling the intervertebral device 140, the spinal surgical procedure can include detaching the intervertebral device 140 from the deployment shaft 144 (FIG. 1K) and closing the proximal portion 141 of the intervertebral device 140. For example, FIG. 1R is an enlarged side view (e.g., a posteriorly-facing view) of a portion of the spine 100 illustrating an intervertebral device closure step of the spinal surgical procedure in accordance with embodiments of the present technology. In the illustrated embodiment, the deployment shaft 144 has been detached from the proximal portion 141 of the intervertebral device 140 and a closure mechanism 146 has been secured to the proximal portion 141 of the intervertebral device 140 such that the filaments 142 and the closure mechanism 146 maintain the fill material 160 within the intervertebral device 140 and inhibit or even prevent egress of the fill material 160 out of the intervertebral device 140. In the illustrated embodiment, the closure mechanism 146 is a nut or screw that is secured within a corresponding opening 145 in the proximal portion 141 of the intervertebral device 140. The closure mechanism 146 can be advanced and actuated (e.g., rotated) by a separate shaft or instrument inserted through the cannula 114 of the trocar 110. In some embodiments, a counter-torque and/or anti-rotation feature is used to allow threading and unthreading of the closure mechanism 146. In other embodiments, the closure mechanism 146 can be a clip, slider, self-closing valve, and/or other mechanism.

After filling and closing the intervertebral device 140, the trocar 110 can be removed from the patient. Referring to FIGS. 1A-1R together, in some aspects of the present technology each of the steps of the spinal surgical procedure can be performed through the minimally-invasive port/access pathway provided by the cannula 114 of the trocar 110. That is, for example: (i) the diseased disc 104a can be accessed and at least partially removed through the cannula 114 of the trocar 110, (ii) the upper vertebra 102a can be lifted and the ligamentous ring 108 further disrupted via the first balloon 130 through the cannula 114 of the trocar 110, (iii) the intervertebral device 140 can be advanced through and deployed from the cannula 114 of the trocar 110 within the disc space 101, (iv) the intervertebral device 140 can be expanded within the disc space 101 via the second balloon 150 through the cannula 114 of the trocar 110, (v) the intervertebral device 140 can be filled with the fill material 160 through the cannula 114 of the trocar 110, and (vi) the intervertebral device 140 can be closed to secure the fill material 160 through the cannula 114 of the trocar 110. Accordingly, embodiments of the present technology can minimize disruption to the flesh of the patient undergoing the surgical procedure—minimizing patient pain and recovery time.

In some embodiments, after removing the trocar 110 from the patient, the spinal surgical procedure further includes attaching a posterior fixation assembly to the spine 100 of the patient. For example, FIG. 1S is a side view (e.g., an anteriorly-facing view) of a portion of the spine 100 of the patient illustrating a posterior fixation step of the spinal surgical procedure in accordance with embodiments of the present technology. In the illustrated embodiment, a posterior fixation assembly 170 is fixedly attached to the upper and lower vertebrae 102a-b to, for example, substantially stabilize the upper and lower vertebrae 102a-b relative to one another. The posterior fixation assembly 170 can include one or more first fixation members 172a secured within the upper vertebra 102a and one or more second fixation members 172b secured within the lower vertebra 102b. The upper and lower fixation members 172a-b can be pedicle screws, cortical screws, wires, bands, interspinous clamps, interlaminar clamps, plates, dowels, and/or the like. Pairs of the upper and lower fixation members 172a-b can be secured together via spanning members 174, such as rods, wires, bands, plates, clamps, and/or the like. The stabilization provided by the posterior fixation assembly 170 can promote bone ingrowth into the intervertebral device 140 (FIGS. 1I-1R).

Referring to FIGS. 1A-1R together, some steps of the spinal surgical procedure can be omitted and/or the various steps can be performed in a different order. For example, the posterior fixation step can be omitted, the balloon expansion step can occur before the mechanical discectomy step, the mechanical discectomy step can be omitted if sufficient disc material is removed via the balloon expansion step, and so on.

FIG. 2 is a flow diagram of a process or method 280 for performing a spinal surgical procedure, such as the spinal surgical procedure (e.g., a spinal fusion procedure) illustrated with reference to FIGS. 1A-1S, in accordance with embodiments of the present technology. At block 281, the method 280 can include inserting a trocar into a patient to proximate a diseased disc via a transpedicular or transforaminal approach, such as described in detail above with reference to FIGS. 1A and 1B. At blocks 282 and 283, the method 280 can include inserting a discectomy device through the trocar and using the mechanical discectomy device to disrupt and/or clear some or all of the diseased disc to form a disc space, respectively, such as described in detail above with reference to FIGS. 1C and 1D. At blocks 284 and 285, the method 280 can include inserting a balloon through the trocar into the disc space and expanding the balloon to further disrupt and/or clear any remaining portion of the diseased disc and to lift an upper vertebra adjacent the diseased disc relative to a lower vertebra adjacent the diseased disc, respectively, as described in detail with reference to FIGS. 1E-1H. In some embodiments, blocks 285 and 286 can be performed before blocks 283 and 284.

At blocks 286 and 287, the method 280 can include inserting an intervertebral device through the trocar into the disc space and expanding the intervertebral device within the disc space, respectively, as described in detail with reference to FIGS. 1I-1K. At block 288, the method 280 can include filling the intervertebral device with a fill material, as described in detail above with reference to FIGS. 1L-1Q. In some embodiments, the same or a different balloon is used to expand the intervertebral device within the disc space at block 287 while, in other embodiments, filling the intervertebral device with the fill material at block 288 can expand the intervertebral device.

At block 289, the method 280 can include tensioning the intervertebral device to, for example, pack the fill material within the intervertebral device. At block 290, the method 280 can include closing the intervertebral device such that the fill material remains therein, as described in detail above with reference to FIG. 1R. At block 291, the method 280 can include releasing the intervertebral device within the disc space by, for example, detaching the intervertebral device from a delivery shaft. Finally, at block 291 the method 280 can include posteriorly fixing the upper and lower vertebrae to stabilize the upper and lower vertebrae.

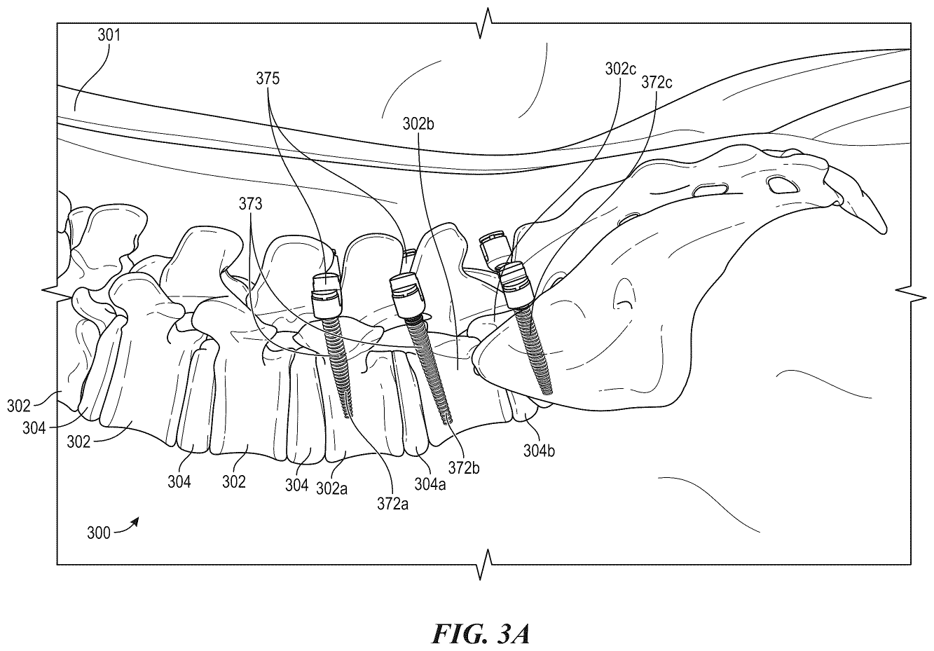

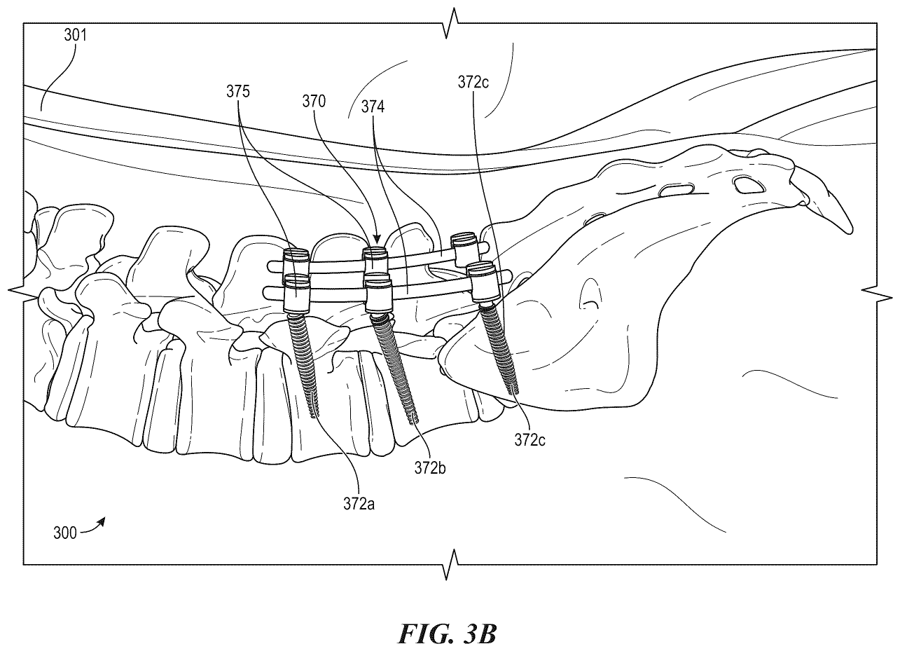

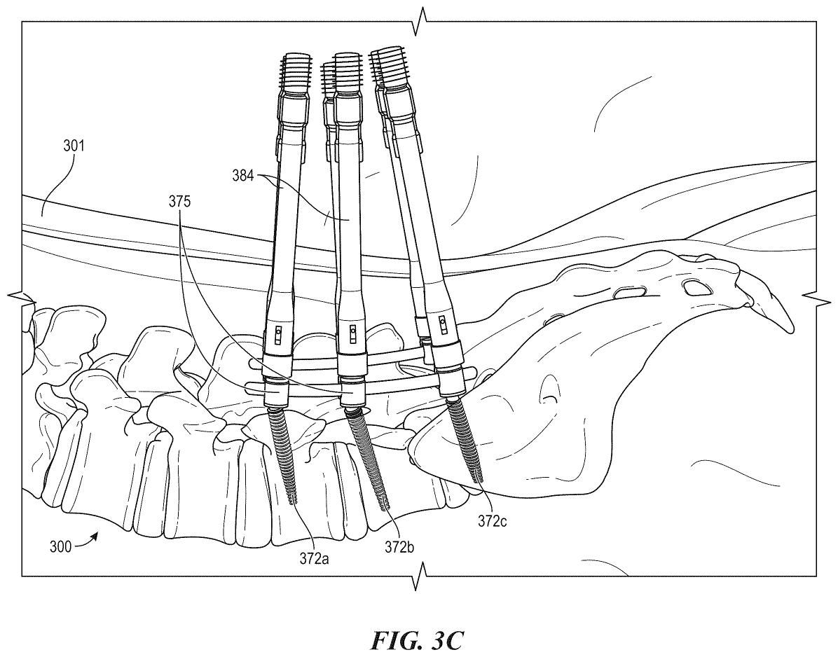

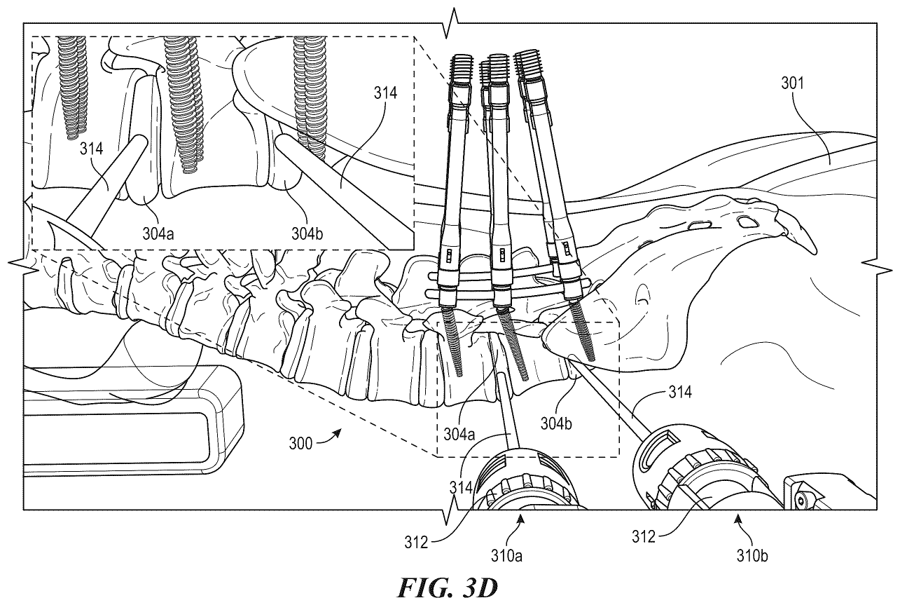

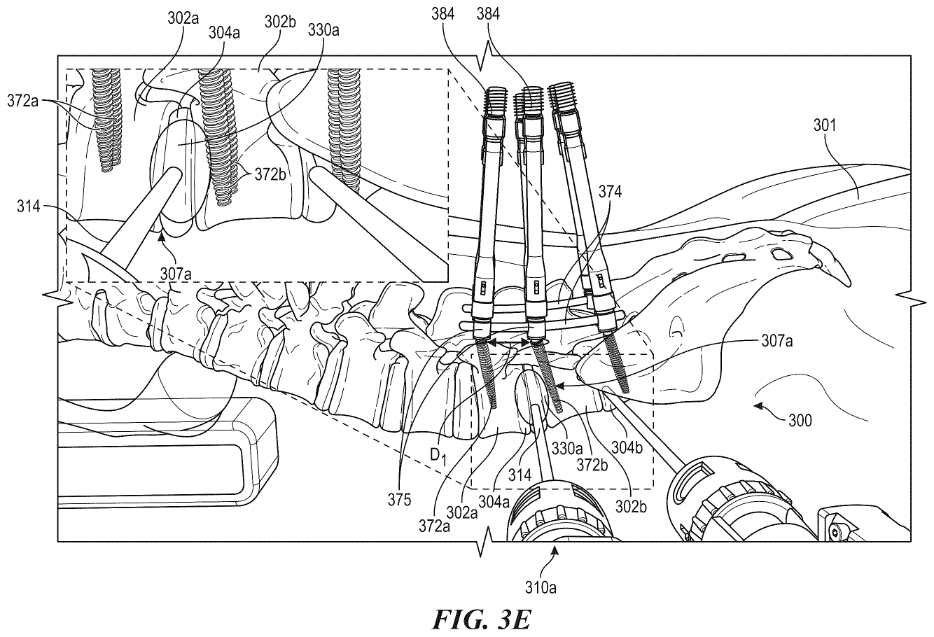

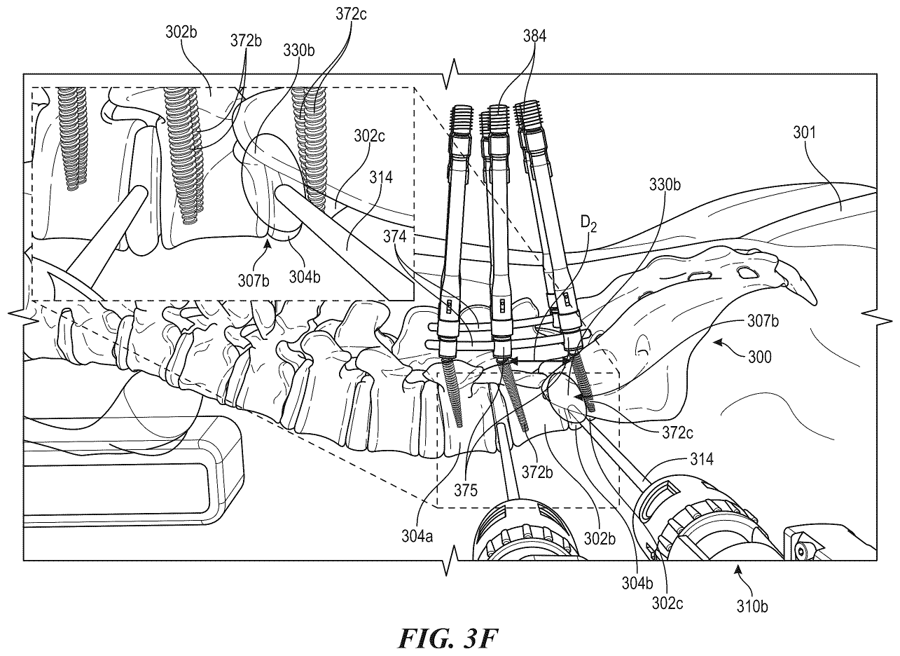

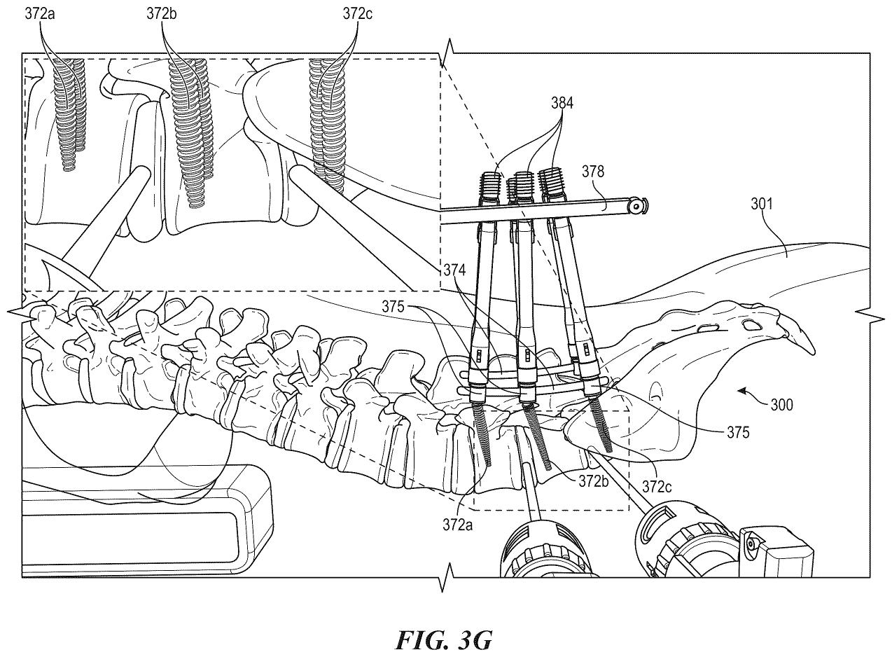

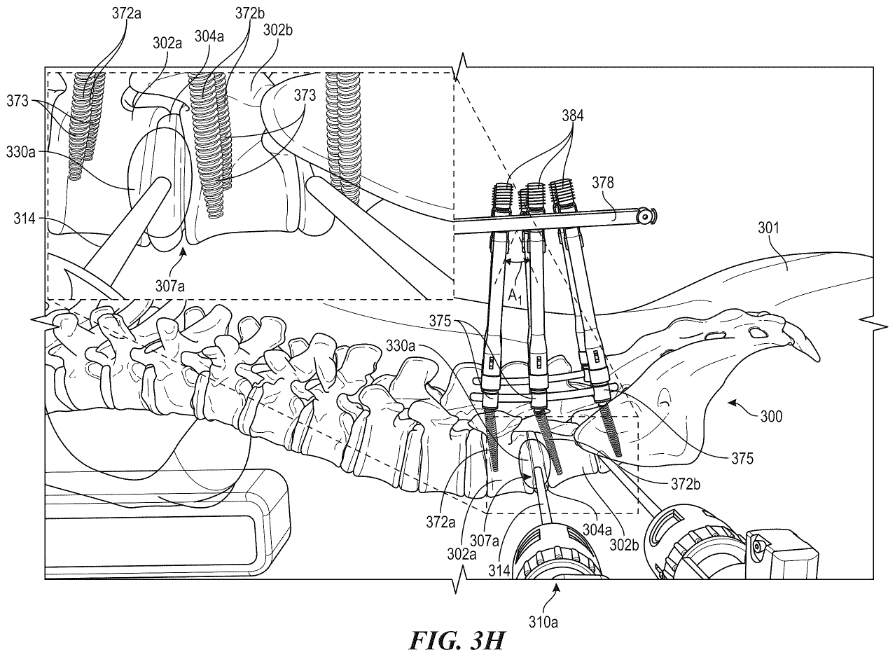

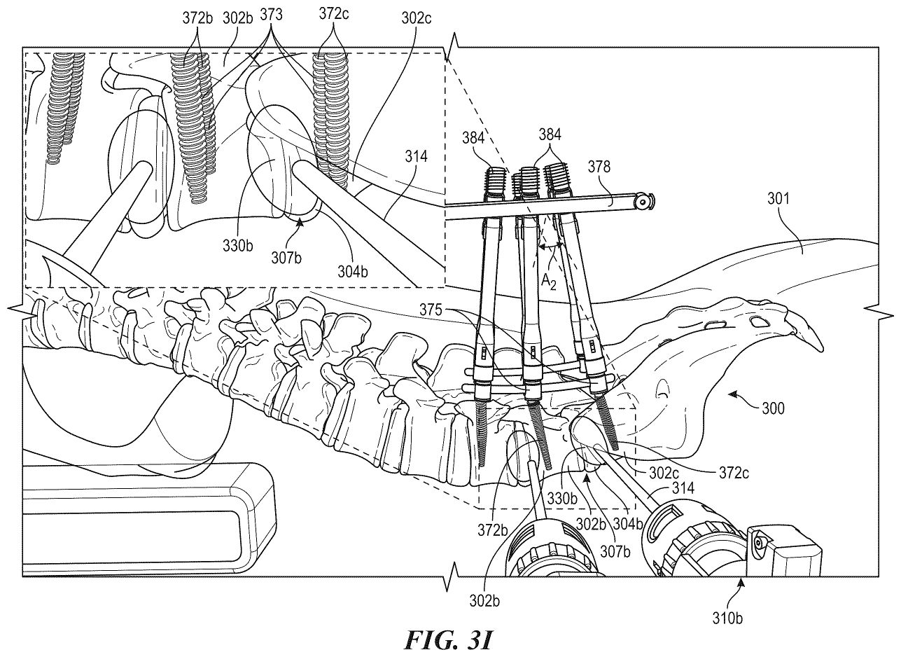

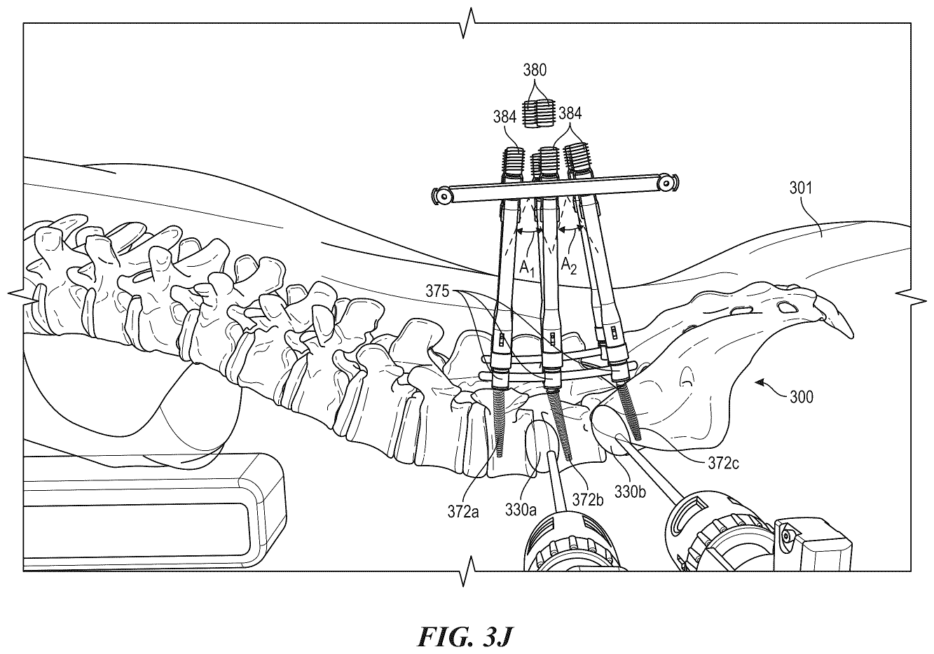

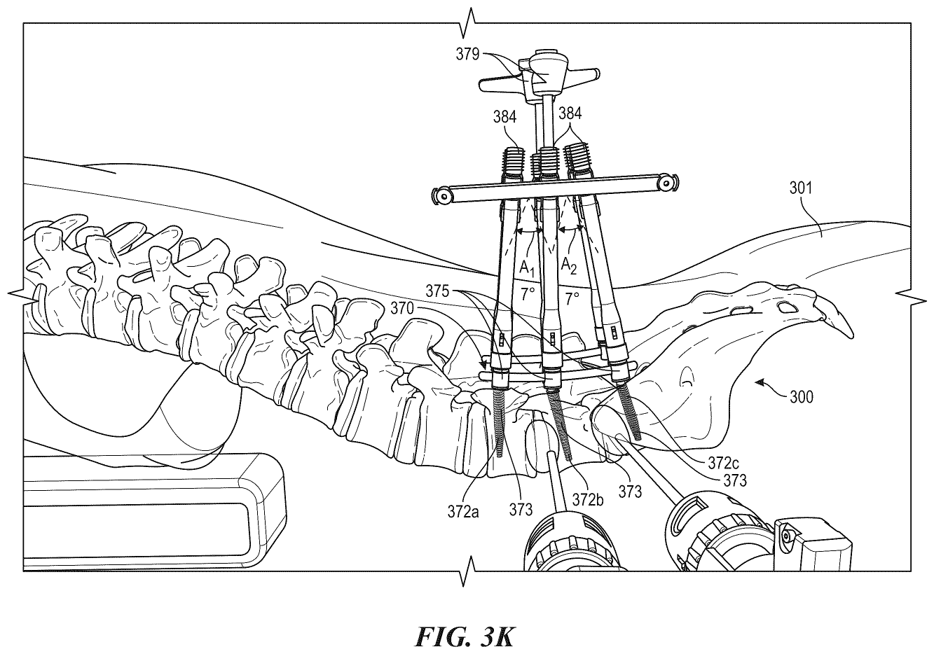

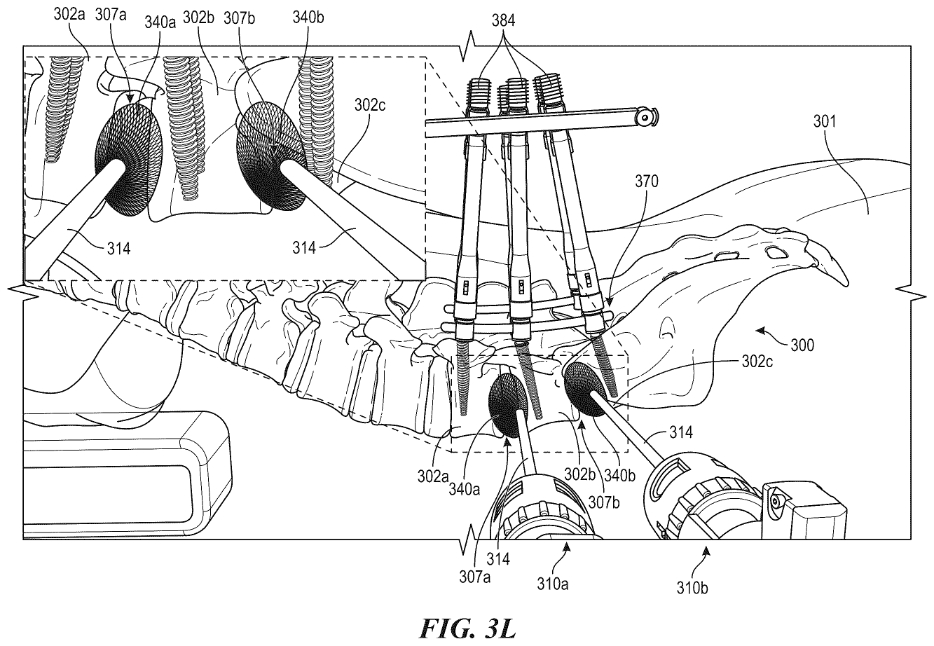

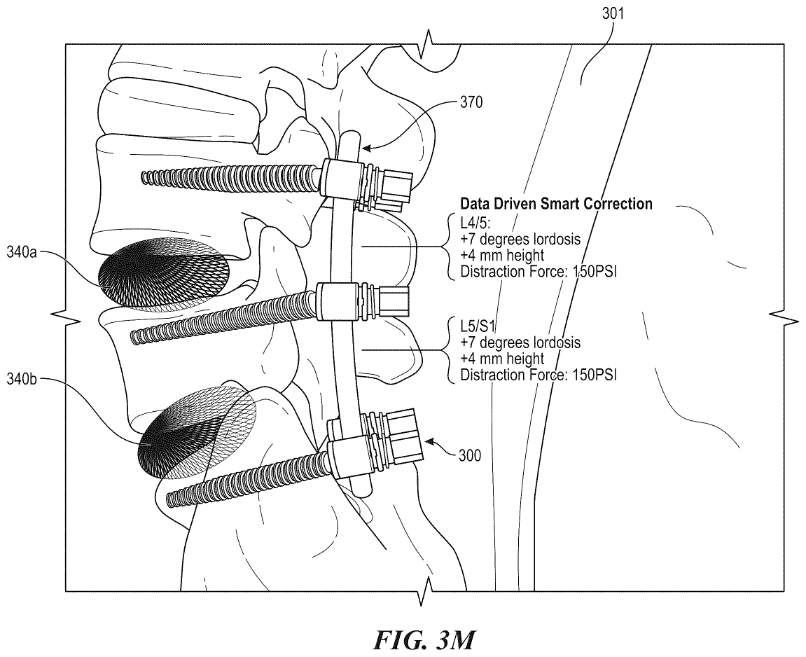

FIGS. 3A-3M are different views of a spinal surgical procedure (e.g., a spinal surgical method) on a spine 300 of a patient 301 (shown as partially transparent for clarity) in accordance with additional embodiments of the present technology. The spinal surgical procedure can be a two-level spinal fusion procedure in which two existing diseased discs of the spine are fully or partially removed, and in which two intervertebral devices are inserted into the disc space to support the adjacent vertebrae and provide for bone ingrowth therein. In other embodiments, the spinal surgical procedure can be a single-level spinal fusion procedure, or a multi-level (e.g., more than two-level) spinal fusion procedure. FIGS. 3A-3M provide an overview of some general aspects/steps of the spinal surgical procedure, and FIGS. 1A-2 and 4A-16 illustrate additional embodiments and/or aspects of the various steps, devices, and/or systems that can be used therein. In some embodiments, some of the steps of the spinal surgical procedure and/or the devices and systems used therein illustrated in FIGS. 3A-3M can include some features that are at least generally similar in structure and function, or identical in structure and function, to the corresponding features of the devices, systems, and/or methods described in detail above with reference to FIGS. 1A-2.

FIG. 3A is a side view (e.g., a lateral view) of a portion of the spine 300 illustrating a first posterior fixation step (e.g., screw insertion step) of the spinal surgical procedure in accordance with embodiments of the present technology. The spine 300 includes a plurality of vertebrae 302 (including an individually identified first or upper vertebra 302a, a second or middle vertebra 302b, and a third or lower vertebra 302c) separated by discs 304 (e.g., intervertebral discs; including an individually identified first diseased disc 304a and a second diseased disc 304b). The upper, middle, and lower vertebrae 302a-c are shown as partially transparent in FIGS. 3A-3M for clarity. The diseased discs 304a-b can result from degenerative joint disease and/or disc arthropathy. In the illustrated embodiment, the upper vertebra 302a is the L4 lumbar vertebra, the middle vertebra is the L5 lumbar vertebra, and the lower vertebra 302c is the S1 sacral vertebra.

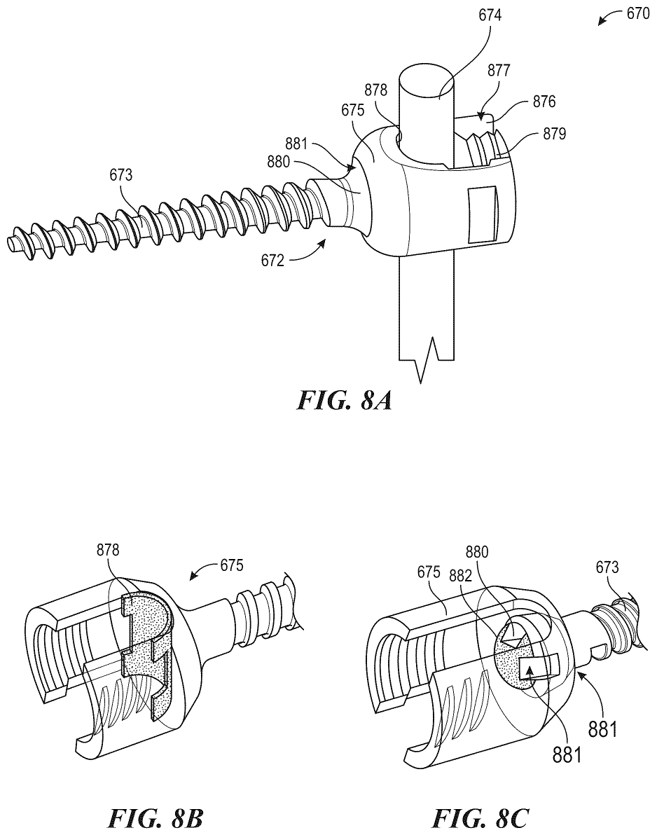

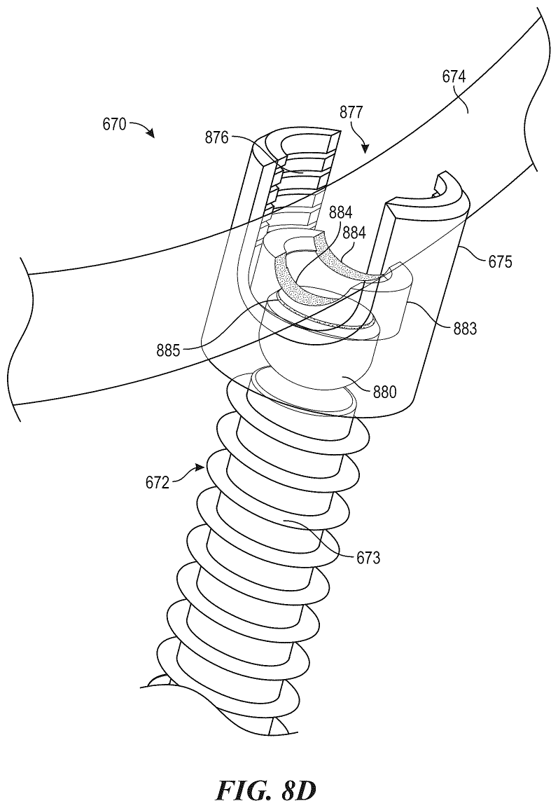

In the first posterior fixation step, one or more (e.g., two) first fixation members 372a are secured within the upper vertebra 302a, one or more (e.g., two) second fixation members 372b are secured within the middle vertebra 302b, and one or more (e.g., two) third fixation members 372c are secured within the lower vertebra 302c. The first, second, and third fixation members 372a-c (collectively “fixation members 372”) can be pedicle screws, cortical screws, wires, bands, interspinous clamps, interlaminar clamps, plates, dowels, and/or the like. For example, in the illustrated embodiment the fixation members 372 are pedicle screws each including a threaded screw body 373 configured to be screwed into and secured within the corresponding one of the vertebrae 302 and a polyaxial head or tulip 375 rotatably coupled to the screw body 373. In some embodiments, the fixation members 372 can include some features similar and/or identical in structure and/or function to the fixation member 1572 described in detail with reference to FIGS. 8A-8D.

FIG. 3B is a side view (e.g., a lateral view) of a portion of the spine 300 illustrating a second posterior fixation step (e.g., a rod insertion step) of the spinal surgical procedure in accordance with embodiments of the present technology. In the second posterior fixation step, one or more spanning members 374 can be coupled to the fixation members 372 to secure the fixation members 372 together. The spanning members 374 can be rods, wires, bands, plates, clamps, and/or the like. In the illustrated embodiment, there are two of the spanning members 374 each comprising a rod, and individual ones of the spanning members 374 are coupled to a corresponding tulip 375 of one of the first fixation members 372a, one of the second fixation members 372b, and one of the third fixation members 372c. The fixation members 372 and the spanning members 374 can together define/comprise a posterior fixation assembly 370.

FIG. 3C is a side view (e.g., a lateral view) of a portion of the spine 300 illustrating a third posterior fixation step (e.g., a tower insertion step) of the spinal surgical procedure in accordance with embodiments of the present technology. In the third posterior fixation step, tower members 384 (e.g., towers, positioning tubes, access channels) can be releasably secured (e.g., rigidly) to corresponding ones of the tulips 375 of the fixation members 372. The tower members 384 can each provide an access channel for accessing a corresponding one of the fixation members 372 during subsequent steps of the spinal surgical procedure described in detail below.

FIG. 3D is a side view (e.g., a lateral view), including an enlarged portion, of a portion of the spine 300 illustrating an access step of the spinal surgical procedure in accordance with embodiments of the present technology. In the access step, a first trocar 310a can be used to access the first diseased disc 304a and a second trocar 310b can be used to access the second diseased disc 304b. In some embodiments, the first and second trocars 310a-b (collectively “trocars 310”) can be inserted via a minimally-invasive lateral approach. In other embodiments, one or both of the trocars 310 can be inserted via a transpedicular or transforaminal (e.g., transfacet) approach such as, for example, described in detail above with reference to FIGS. 1A and 1B. In yet other embodiments, one or both of the trocars 310 can be inserted via an anterior approach. The trocars 310 can include some features generally similar or identical in structure and/or function to the trocar 110 described in detail above with reference to FIGS. 1A-1S and/or elsewhere herein. For example, in the illustrated embodiment, the trocars 310 each include a handle 312 coupled to a hollow cannula 314 defining a lumen. In some embodiments, an introducer is positioned within the lumens of each of the trocars 310 during the access step as the trocars 310 are pushed, rotated, and/or otherwise advanced through the flesh of the patient 301 to proximate the diseased discs 304a-b.

FIGS. 3E and 3F are side views (e.g., lateral views), including enlarged portions, of a portion of the spine 300 illustrating distraction steps (e.g., a parallel distraction steps) of the spinal surgical procedure in accordance with embodiments of the present technology. Referring to FIG. 3E, a first balloon 330a can be inserted through the cannula 314 of the first trocar 310a and inflated in a first disc space 307a between the upper vertebra 302a and the middle vertebra 302b to distract the first disc space 307a and create separation (e.g., height) between the upper and middle vertebrae 302a-b. For example, inflation of the first balloon 330a can force the upper and middle vertebrae 302a-b to move away from one another by a first distance D₁ of between about 1-15 millimeters, between about 1-10 millimeters, between about 1-8 millimeters, about 8 millimeters, etc. The first fixation members 372a and the second fixation members 372b (e.g., the screw bodies 373 thereof; FIG. 3A) are fixed within the upper and lower vertebrae 302a-b, respectively, such that they move therewith during expansion of the first balloon 330a. Accordingly, in some embodiments the tulips 375 (and coupled tower members 384) of the first fixation members 372a and/or the tulips 375 of the second fixation members 372b can slide along the spanning members 374 during expansion of the first balloon 330a.

Referring to FIG. 3F, a second balloon 330b can similarly be inserted through the cannula 314 of the second trocar 310b and inflated in a second disc space 307b between the middle vertebra 302b and the lower vertebra 302c to distract the second disc space 307b and create separation (e.g., height) between the middle and lower vertebrae 302b-c. For example, inflation of the second balloon 330b can force the middle and lower vertebrae 302b-c to move away from one another by a first distance D₂ of between about 1-15 millimeters, between about 1-10 millimeters, between about 1-8 millimeters, about 8 millimeters, etc. The second fixation members 372b and the third fixation members 372c (e.g., the screw bodies 373 thereof; FIG. 3A) are fixed within the middle and lower vertebrae 302b-c, respectively, such that they move therewith during expansion of the second balloon 330b. Accordingly, in some embodiments the tulips 375 (and coupled tower members 384) of the second fixation members 372b and/or the tulips 375 of the third fixation members 372c can slide along the spanning members 374 during expansion of the second balloon 330b.

Referring to FIGS. 3E and 3F, the first and second balloons 330a-b can include some features generally similar or identical in structure and/or function to those of the first balloon 130 described in detail above with reference to FIGS. 1E-1H and/or elsewhere herein. In some embodiments, a discectomy device is first inserted through the first trocar 310a to remove some or all of the first diseased disc 304a before distraction of the first disc space 307a with the first balloon 330a and/or the same or a different discectomy device is first inserted through the second trocar 310b to remove some or all of the second diseased disc 304b before distraction of the second disc space 307b with the second balloon 330b as, for example, described in detail above with reference to FIGS. 1C and 1D and/or elsewhere herein. The balloons 330a-b can be expanded sequentially (e.g., the first balloon 330a before the second balloon 330b, the second balloon 330b before the first balloon 330a) or simultaneously.

FIGS. 3G-3I are side views (e.g., lateral views), including enlarged portions, of a portion of the spine 300 illustrating distraction steps (e.g., a lordosis and distraction steps) of the spinal surgical procedure in accordance with additional embodiments of the present technology. The distraction steps illustrated in FIGS. 3G-3I can be performed as an alternative to the distraction steps illustrated in FIGS. 3E and 3F—or can be performed after the distraction steps illustrated in FIGS. 3E and 3F.

Referring to FIG. 3G, a locking device 378 (e.g., a positioning tie) can be releasably secured to some or all of the tower members 385. The locking device 378 can be a clamp or similar structure configured to fixedly secure (e.g., lock) the position and orientation of the tower members 385 relative to one another. The tower members 385 are fixedly coupled to corresponding ones of the tulips 375 of the fixation members 372 such that the locking device 378 further acts to fixedly secure (e.g., lock) the position and orientation of the tulips 375 relative to another. That is, for example, the locking device 378 can inhibit or even prevent the tulips 375 from sliding (e.g., axially) along the spanning members 374 and/or rotating. In some embodiments, the locking device 378 can comprise one or more clips, clamps, and/or the like that are fixed to the spanning members 374 adjacent the tulips 375 to inhibit or even prevent the tulips 375 from sliding (e.g., axially) along the spanning members 374. More generally, the locking device 378 is configured to inhibit or even prevent (e.g., lock) axial movement of the tulips 375 along the spanning members 374 such that the fixation members 372 are constrained to pivot rather than move laterally relative to one another.

Referring to FIG. 3H, the first balloon 330a can be inserted through the cannula 314 of the first trocar 310a and inflated in the first disc space 307a between the upper vertebra 302a and the middle vertebra 302b to distract the first disc space 307a and create separation (e.g., height) and lordosis between the upper and middle vertebrae 302a-b. For example, inflation of the first balloon 330a can force the upper and middle vertebrae 302a-b to pivot away from one another by an angle A₁ of between about 1-15 degrees, between about 1-10 degrees, between about 2-8 degrees, about 7 degrees, etc. More specifically, the locking device 378 can fixedly secure the position and orientation of the tulips 375 relative to one another such that the threaded screw bodies 373 (labeled in the enlarged portion of the view) of the first and second fixation members 372a-b are constrained to pivot about/within the tulips 375. Such mechanical constraint of the first and second fixation members 372a-b constrains the upper and middle vertebrae 302a-b to pivot to create the angle A₁ as the first balloon 330a is expanded as opposed to simply moving laterally away from another as shown in, for example, FIG. 3E. In some embodiments, the tower members 384 and/or another component of the system can include one or more devices configured to measure the angle A₁ in real time or near real time to, for example, provide intelligent feedback to a surgeon or other operator of the effect of the expansion of the first balloon 330a on the curvature of the spine 300. Such devices for measuring the angle A₁ can be optical, electrical, mechanical, and/or the like.

Referring to FIG. 3I, the second balloon 330b can similarly be inserted through the cannula 314 of the second trocar 310b and inflated in the second disc space 307b between the middle vertebra 302b and the lower vertebra 302c to distract the second disc space 307b and create separation (e.g., height) and lordosis between the middle and lower vertebrae 302b-c. For example, inflation of the second balloon 330b can force the middle and lower vertebrae 302b-c to pivot away from one another by an angle A₂ of between about 1-15 degrees, between about 1-10 degrees, between about 2-8 degrees, about 7 degrees, etc. More specifically, the locking device 378 can fixedly secure the position and orientation of the tulips 375 relative to one another such that the threaded screw bodies 373 (labeled in the enlarged portion of the view) of the second and third fixation members 372b-c are constrained to pivot about/within the tulips 375. Such mechanical constraint of the second and third fixation members 372b-c constrains the middle and lower vertebrae 302b-c to pivot to create the angle A₂ as the second balloon 330b is expanded as opposed to simply moving laterally away from another as shown in, for example, FIG. 3F. In some embodiments, the tower members 384 and/or another component of the system can include one or more devices configured to measure the angle A₂ in real time or near real time to, for example, provide intelligent feedback to a surgeon or other operator of the effect of the expansion of the first balloon 330a on the curvature of the spine 300. Such devices for measuring the angle A₂ can be optical, electrical, mechanical, and/or the like. In some embodiments, the angle A₂ can be the same as or similar to the angle A₁ (FIG. 3H).

Referring to FIGS. 3H and 3I, the first and second balloons 330a-b can include some features generally similar or identical in structure and/or function to those of the first balloon 130 described in detail above with reference to FIGS. 1E-1H and/or elsewhere herein. In some embodiments, a discectomy device is first inserted through the first trocar 310a to remove some or all of the first diseased disc 304a before distraction of the first disc space 307a with the first balloon 330a and/or the same or a different discectomy device is first inserted through the second trocar 310b to remove some or all of the second diseased disc 304b before distraction of the second disc space 307b with the second balloon 330b as, for example, described in detail above with reference to FIGS. 1C and 1D and/or elsewhere herein. The balloons 330a-b can be expanded sequentially (e.g., the first balloon 330a before the second balloon 330b, the second balloon 330b before the first balloon 330a) or simultaneously.

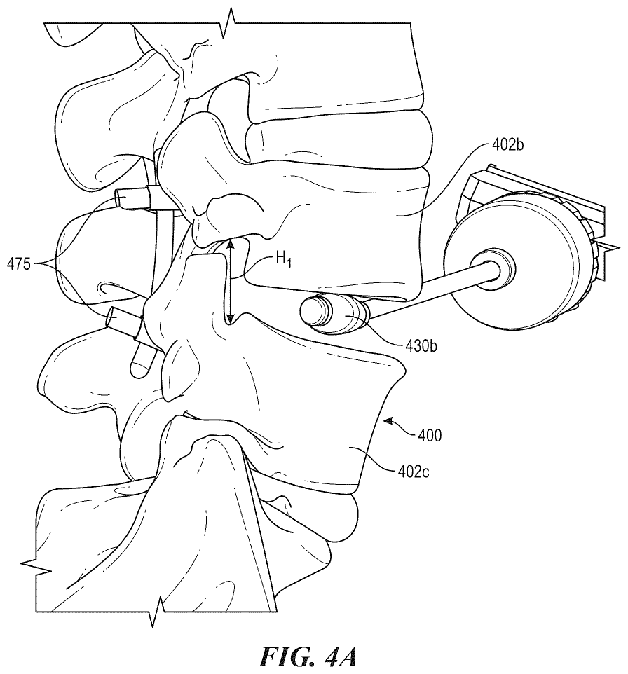

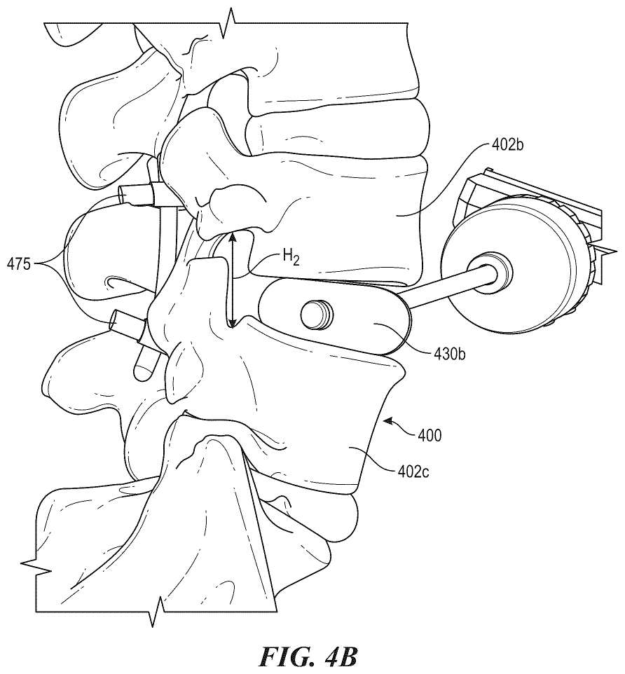

In some aspects of the present technology, inflation of the first and second balloons 330a-b can create lordosis of the spine 300 without compressing the foramen around the nerve root and, in some embodiments, can decompress the foramen around the nerve root. For example, the pivot points of the vertebrae 302 at the posterior fixation assembly 370 (e.g., at the tulips 375) are located behind (e.g., posterior to) the foramen and the nerve root such that inflation of the first and second balloons 330a-b increases the intervertebral foraminal height. More specifically, FIGS. 4A and 4B are side views (e.g., lateral views) of a portion of the spine 300 before and after inflation of the second balloon 330b in accordance with embodiments of the present technology. Referring to FIG. 4A, before inflation of the second balloon 330b the middle and lower vertebrae 302b-c can have/define a first foraminal height H₁. Referring to FIG. 4B, inflation of the second balloon 330b can create lordosis of the spine 300 and increase the height to a second foraminal height H₂ greater than the first foraminal height H₁. This can reduce compression around a nerve root extending from the foramen and is achieved because the pivot point for lordosis creation is at the tulips 375 positioned behind (e.g., posterior to) the foramen. In contrast, many conventional surgical techniques utilizing an interbody create lordosis by compressing the screws pivoting on the anterior edge. If the facet joint is not left intact and then the segments are semi-free floating during placement of the interbody and the posterior is reduced to create the angle, the foraminal height is reduced, potentially creating compression of the nerve root.

FIGS. 3J and 3K are side views (e.g., lateral views) of a portion of the spine 300 illustrating posterior fixation locking steps of the spinal surgical procedure in accordance with embodiments of the present technology. The posterior fixation steps illustrated in FIGS. 3J and 3K can be performed after the distraction and lordosis steps illustrated in FIGS. 3G-3I. Referring to FIG. 3J, with the first balloon 330a and the second balloon 330b expanded to maintain the lordotic angles A₁ and A₂, a set screw 380 can be inserted through each of the tower members 384 and into the corresponding one of the tulips 375 of the fixation members 372. Referring to FIG. 3J, with the first balloon 330a and the second balloon 330b expanded to maintain the lordotic angles A₁ and A₂, the posterior fixation assembly 370 can be locked in position by inserting one or more drivers 379 through the tower members 384 and rotating the driver(s) 379 to tighten the set screws 380 (FIG. 3J) to, for example, lock (e.g., via friction) an orientation/position of the tulip 375 relative to (i) the screw body 373 of each of the fixation members 372 and (ii) the spanning members 374. In some embodiments, the fixation members 372 can be locked in position/orientation as described in detail below with reference to FIGS. 8A-8D and/or elsewhere herein.

FIG. 3L is a side view (e.g., a lateral view), including an enlarged portion, of a portion of the spine 300 illustrating an intervertebral device deployment step of the spinal surgical procedure in accordance with embodiments of the present technology. In the illustrated embodiment, after locking the posterior fixation assembly 370 (FIG. 3K), the first and second balloons 330a-b can be deflated and removed through the cannulas 314 of the first and second trocars 310a-b, respectively, and (i) a first intervertebral device 340a can be inserted through the cannula 314 of the first trocar 310a and deployed within the first disc space 307a between the upper and middle vertebrae 302a-b and (ii) a second intervertebral device 340b can be inserted through the cannula 314 of the second trocar 310b and deployed within the second disc space 307b between the middle and lower vertebrae 302b-c. In some embodiments, the first and second intervertebral devices 340a-b can include some features generally similar or identical in structure and/or function to the intervertebral device 140 described in detail above with reference to FIGS. 1I-1R and/or elsewhere herein, and can be deployed in a generally similar or identical manner (e.g., including expanding, filling, tensioning, and closing). In some embodiments, a discectomy device is first inserted through the first trocar 310a to remove some or all of the first diseased disc 304a (FIG. 3A) before deployment of the first intervertebral device 340a and/or the same or a different discectomy device is first inserted through the second trocar 310b to remove some or all of the second diseased disc 304b (FIG. 3A) before deployment of the second intervertebral device 340b as, for example, described in detail above with reference to FIGS. 1C and 1D and/or elsewhere herein. Alternatively, the first and second intervertebral devices 340a-b can be deployed after the distraction steps illustrated in FIGS. 3E and 3F without locking of the posterior fixation assembly 370. The posterior fixation assembly 370 can then be locked after deployment of the first and second intervertebral devices 340a-b.

Finally, the tower members 384 and the first and second trocars 310a-b can be removed from the patient. FIG. 3M, for example, is a side view of a portion of the spine 300 illustrating the finally-implanted first and second intervertebral devices 340a-b and the posterior fixation assembly 370 in accordance with embodiments of the present technology.

Referring to FIGS. 3A-3M, in some aspects of the present technology the spinal surgical procedure can be performed without the use of retractors—as the first and second trocars 310a-b provide a minimally-invasive, retractor-less access port for the deployment of the of the first and second intervertebral devices 340a-b, distraction and lordosis of the first and second disc spaces 307a-b, etc. Not requiring the use of retractors can minimize trauma to muscle, viscera, nerves, and/or the like of the patient 301, which is a significant contributor to non-trivial post-operative pain in conventional spinal surgical procedures. Likewise, in additional aspects of the present technology the patient 301 can be positioned in a single position (e.g., a prone position) during the entirety of the spinal surgical procedure. In particular, the trocars 310a-b can provide an access port to the first and second disc spaces 307a-b that does not require direct visualization such that the patient 301 can be positioned in a single position during installation and manipulation of the posterior fixation assembly 370. Moreover, although a two-level spinal fusion procedure is illustrated in FIGS. 3A-3M, the spinal surgical procedure can be similarly carried out to treat only a single diseased one of the discs 304 and to fuse only two adjacent levels of the vertebrae 302, and/or to treat more than two diseased one of the discs 304 and to fuse more than three adjacent levels of the vertebrae 302.

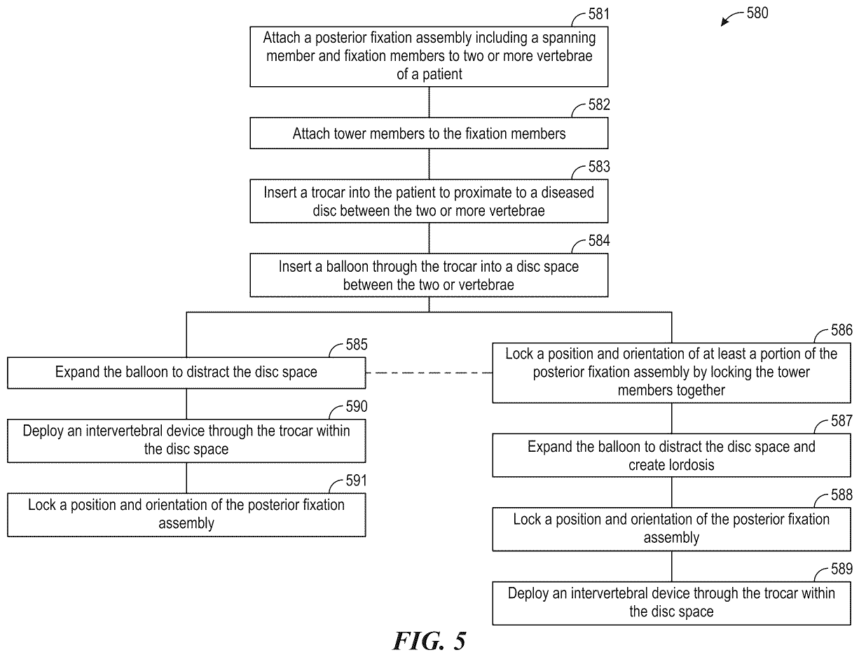

FIG. 5 is a flow diagram of a process or method 580 for performing a spinal surgical procedure, such as the spinal surgical procedure (e.g., a spinal fusion procedure) illustrated with reference to FIGS. 3A-3M, in accordance with embodiments of the present technology. At block 581, the method 580 can include attaching a posterior fixation assembly including at least one spanning member and fixation members to two or more vertebrae of a patient, such as described in detail above with reference to FIGS. 3A and 3B. At block 581, the method 580 can include attaching tower members to the fixation members, such as described in detail above with reference to FIG. 3C. At block 583, the method 580 can include inserting at least one trocar into the patient to proximate a diseased disc between the two or more vertebrae via, for example, a lateral approach, such as described in detail above with reference to FIG. 3D. In some embodiments, such as for a two-level fusion procedure, a first trocar is inserted to proximate a first diseased disc and a second trocar is inserted to proximate a second diseased disc. At block 584, the method can include inserting a balloon through the trocar into a disc space between the two or more vertebrae. In some embodiments, such as for a two-level fusion procedure, a first balloon is inserted into a first disc space via the first trocar and a second balloon is inserted into a second disc space via the second trocar.

After block 584, the method 580 can proceed to block 585 to include expanding the balloon(s) to distract the disc space(s), as described in detail above with reference to FIGS. 3E and 3F, or to block 586 to lock the position and orientation of at least a portion of the posterior fixation assembly (e.g., tulips of the fixation members) by locking the tower members together, as described in detail above with reference to FIG. 3G. In some embodiments, the method 580 can proceed from block 585 to block 586. At block 587, the method 580 can include expanding the balloon(s) to distract the disc space(s) and create lordosis of the two or more vertebrae. In some embodiments, the method 580 includes determining/measuring the created lordosis (e.g., lordotic angle) in real time or near real time.

At block 588, the method 580 can include locking the orientation and position of the posterior fixation assembly, as described in detail above with reference to FIGS. 3J and 3K. At block 589, the method 580 can include deploying an intervertebral device through the trocar within the disc space, as described in detail above with reference to FIG. 3L. In some embodiments, such as for a two-level fusion procedure, a first intervertebral device is deployed within the first disc space via the first trocar and a second intervertebral device is deployed within the second disc space via the second trocar.

If the method proceeds to block 585 and not to block 586, the method 580 can include deploying the intervertebral device(s) within the disc space(s) through the trocar(s) at block 590, and then locking a position and orientation of the posterior fixation assembly at block 591.

FIGS. 6A and 6B are side views (e.g., lateral views) of a portion of a spine 600 illustrating distraction steps (e.g., parallel distraction steps) of a spinal surgical procedure in accordance with embodiments of the present technology. The distraction steps can be similar to, for example, the distraction steps described in detail above with reference to FIGS. 3E and 3F.

The spine 600 includes a plurality of vertebrae 602 (including an individually identified first or upper vertebra 602a and a second or lower vertebra 602b). The upper and lower vertebrae 602a, b are separated by a disc space 607 in which a diseased disc has been at least partially removed. As described in detail above, one or more (e.g., two) first fixation members 672a are secured within the upper vertebra 602a, one or more (e.g., two) second fixation members 672b are secured within the lower vertebra 602b. The first and second fixation members 672a-b (collectively “fixation members 672”) can be pedicle screws, cortical screws, wires, bands, interspinous clamps, interlaminar clamps, plates, dowels, and/or the like. For example, in the illustrated embodiment the fixation members 672 are pedicle screws each including a threaded screw body 673 configured to be screwed into and secured within the corresponding one of the vertebrae 602 and a polyaxial head or tulip 675 rotatably coupled to the screw body 673. Further details of the fixation members 672 are described in detail below with reference to FIGS. 8A-8D. One or more spanning members 674 can be coupled to the fixation members 672 to secure the fixation members 672 together. The spanning members 674 can be rods, wires, bands, plates, clamps, and/or the like. In the illustrated embodiment, there is one of the spanning members 674 comprising a rod, and the spanning member 674 is coupled to the corresponding tulips 675 of the first and second fixation members 672a, b. The fixation members 672 and the spanning member 674 can together define/comprise a posterior fixation assembly 670.

In some embodiments, during the distraction steps, the tulips 675 are not tightened or not fully tightened against the spanning member 674 and the screw bodies 673. Accordingly, the screw bodies 673 can be free to pivot polyaxially relative to the tulips 675 while the spanning member 674 remains slidable through the tulips 675. In the illustrated embodiment, a balloon 630 is inserted into the disc space 607. The balloon 630 can be inserted through an access channel of a trocar (not shown) and inflated to an inflated configuration (FIG. 6B) from an uninflated configurated (FIG. 6A). Inflation of the balloon 630 between the upper and lower vertebrae 602a-b can force the upper and lower vertebrae 602a-b to move away from one another to distract the first disc space 607 and create separation (e.g., height) between the upper and lower vertebrae 602a-b. For example, inflation of the balloon 630 can force the upper and lower vertebrae 602a-b to move away from a first distance D₁ (FIG. 6A) to a second distance D₂ (FIG. 6B), greater than the first distance D₁. A difference between the second distance D₂ and the first distance D₁ (e.g., the created height) can be between about 1-15 millimeters, between about 1-10 millimeters, between about 1-8 millimeters, about 8 millimeters, etc. The first fixation member 672a and the second fixation member 672b (e.g., the screw bodies 673 thereof) are fixed within the upper and lower vertebrae 602a-b, respectively, such that they move therewith during expansion of the balloon 630. Accordingly, in some embodiments the tulip 675 of the first fixation member 672a and/or the tulip 675 of the second fixation member 672b can slide along the spanning members 674 during expansion of the balloon 630.

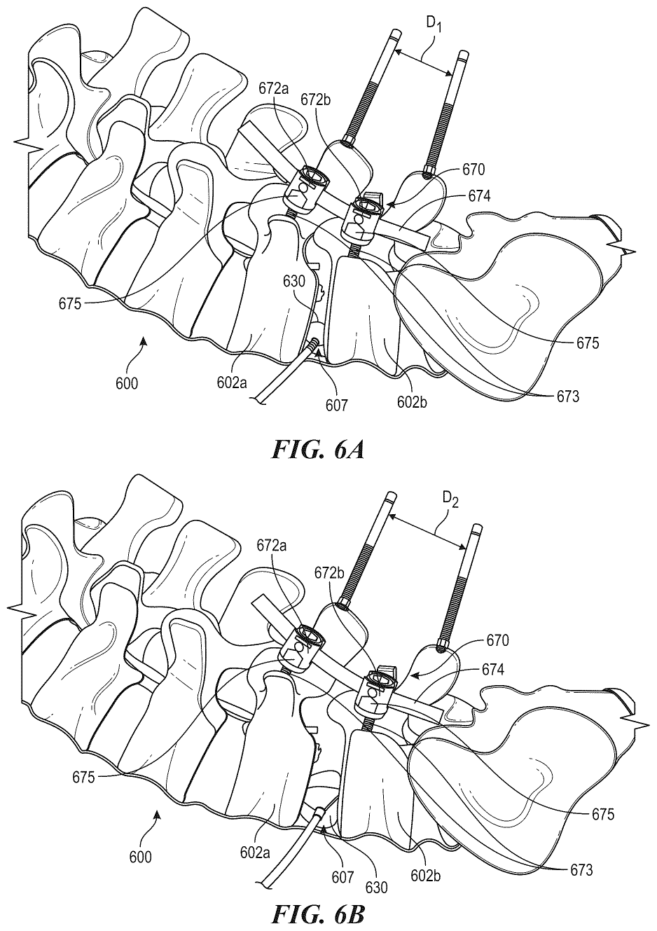

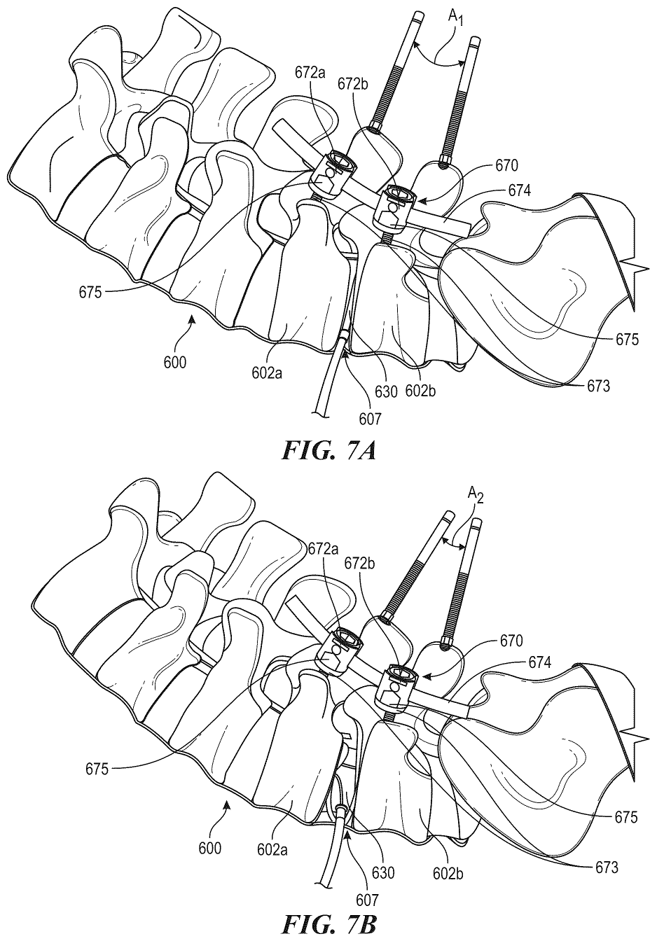

FIGS. 7A and 7B are side views (e.g., lateral views) of the portion of the spine 600 of FIGS. 6A and 6B illustrating distraction steps (e.g., a lordosis and distraction steps) of the spinal surgical procedure in accordance with additional embodiments of the present technology. The distraction steps illustrated in FIGS. 7A and 7B can be performed as an alternative to the distraction steps illustrated in FIGS. 6A and 6B—or can be performed after the distraction steps illustrated in FIGS. 6A and 6B. The distraction steps can be similar to, for example, the distraction steps described in detail above with reference to FIGS. 3G-3I.

In the illustrated embodiment, the balloon 630 is similarly inserted into the disc space 607 and inflated to the inflated configuration (FIG. 7B) from the uninflated configurated (FIG. 7A). However, the fixation members 672 can be in a partially tightened state (e.g., a partially-capped state) that inhibits the tulips 675 from sliding along the spanning member 674 during inflation of the balloon 630 while still permitting the screw bodies 673 to pivot polyaxially within the tulips 675. That is, the fixation members 672 can be at least partially tightened to inhibit or even prevent (e.g., lock) axial movement of the tulips 675 along the spanning member 674 such that the fixation members 672 are constrained to pivot rather than move laterally relative to one another.

Accordingly, expansion of the balloon 630 distracts the disc space 607 and creates separation (e.g., height) and lordosis between the upper and lower vertebrae 602a-b. For example, inflation of the balloon 630 can force the upper and lower vertebrae 602a-b to pivot away from one another from a first angle A₁ (FIG. 7A) to a second angle A₂ (FIG. 7B), greater than the first angle A₁. A difference between the second angle A₂ and the first angle A₁ can be between about 1-15 degrees, between about 1-10 degrees, between about 2-8 degrees, about 7 degrees, etc. More specifically, the partial tightening of the first and second fixation members 672a-b can fixedly secure the position and orientation of the tulips 675 relative to one another by securing the tulips 675 to the spanning member 674, while still permitting for polyaxial movement of the screw bodies 673 within the tulips 675 such that the screw bodies 673 are constrained to pivot about/within the tulips 675. Such mechanical constraint of the first and second fixation members 672a-b constrains the upper and lower vertebrae 602a-b to pivot to create the second angle A₂ as the balloon 630 is expanded as opposed to simply moving laterally away from another as shown in, for example, FIGS. 6A and 6B. Accordingly, the partial tightening of the fixation members 672 can achieve the same effect of lordosis creation during expansion of the balloon 630—as described in detail above with reference to FIGS. 3G-3I—but without the separate locking device 378, rod limiters, and/or the like. Accordingly, the illustrated methodology can more simply utilize existing hardware of the posterior fixation assembly 670 to induce lordosis without additional mechanical devices/constraints.

In some aspects of the present technology, inflation of the balloon 630 as shown in FIG. 7B can create lordosis of the spine 600 without compressing the foramen around the nerve root and, in some embodiments, can decompress the foramen around the nerve root. For example, the pivot points of the vertebrae 602 at the tulips 675 are located behind (e.g., posterior to) the foramen and the nerve root such that inflation of the balloon 630 increases the intervertebral foraminal height. This can reduce compression around a nerve root extending from the foramen and is achieved because the pivot point for lordosis creation is at the tulips 675 positioned behind (e.g., posterior to) the foramen. In contrast, many conventional surgical techniques utilizing an interbody create lordosis by compressing the screws pivoting on the anterior edge. If the facet joint is not left intact and then the segments are semi-free floating during placement of the interbody and the posterior is reduced to create the angle, the foraminal height is reduced, potentially creating compression of the nerve root.

FIG. 8A is a perspective view of a portion of the posterior fixation assembly 670 of FIGS. 7A and 7B including one of the fixation members 672 in accordance with embodiments of the present technology. In the illustrated embodiment, the tulip 675 defines a U-shaped surface 876. The surface 876 at least partially defines/encloses a channel 877 configured to receive the spanning member 674 therethrough. The surface 876 can be at least partially threaded and have a saddle portion 878 (e.g., at the trough thereof). In the illustrated embodiment, a nut 879 is positioned within the channel 877 and threadably engages at least a portion of the surface 876. The screw body 673 (e.g., screw shaft) can include a head 880 (e.g., a generally spherical head) that is rotatably (e.g., polyaxially) positioned within the channel 877 through an aperture 881 in the tulip 675. In the illustrated embodiment, the spanning member 674 is generally cylindrical and extends through the channel 877 between the nut 879 and the head 880.

The nut 879 can be tightened via a driver and/or another torque member to compress the spanning member 674 against the saddle portion 878 of the tulip 675 and against the head 880. FIG. 8B, for example, is a partially-transparent perspective view of the tulip 675 in accordance with embodiments of the present technology. As shown by highlighting, the saddle portion 878 provides a region of contact between the tulip 675 and the spanning member 674 (FIG. 8A). Similarly, FIG. 8C is a partially-transparent perspective view of the tulip 675 and the screw body 673 in accordance with embodiments of the present technology. As shown by highlighting, a contact portion 882 of the head 880 projecting through the aperture 881 provides a region of contact between the spanning member 674 (FIG. 8A) and the head 880. Referring to FIGS. 8A-8C, the saddle portion 878 can be larger than the contact portion 882. Accordingly, when the nut 879 is tightened to increase the compressive force (i) between the saddle portion 878 and the spanning member 674 and (ii) between the contact portion 882 of the head 880 and the spanning member 674, a restraining force (e.g., frictional force) between the spanning member 674 and the tulip 675 can increase at a greater rate than a restraining force (e.g., frictional force) between the spanning member 674 and the head 880.

FIG. 8D is a partially-transparent perspective view of the fixation member 672 and the spanning member 674 of FIGS. 8A-8C in accordance with additional embodiments of the present technology. In the illustrated embodiment, the fixation member 672 further comprises a saddle 883 (e.g., a grooved insert) configured to be positioned between the head 880 of the screw body 673 and the spanning member 674. The tulip 675, the saddle 883, and the spanning member 674 are shown as partially transparent in FIG. 8D for clarity. More specifically, as shown by highlighting, the saddle 883 can define an upper surface contact region 884 configured to contact the spanning member 674 and a lower surface contact region 885 configured to contact the head 880. The saddle 883 can be annular such that the upper surface contact region 884 comprises two spaced-apart U-shaped surfaces, and the lower surface contact region 885 comprises a ring. The upper surface contact region 884 can be larger than the lower surface contact region 885. Accordingly, when the nut 879 (now shown; FIG. 8A) is tightened, a compressive force is increased (i) between the upper surface contact region 884 of the saddle 883 and the spanning member 674 and (ii) between the lower surface contact region 885 of the saddle 883 and the head 880. Due to the differential sizes of the upper and lower surface contact regions 884, 885, a restraining force (e.g., frictional force) between the spanning member 674 and the tulip 675 (e.g., the saddle 883) can increase at a greater rate than a restraining force (e.g., frictional force) between the tulip 675 (e.g., the saddle 883) and the head 880.

Based on this geometry, referring to FIGS. 7A-8D, the nut 879 can be tightened to a partially-tightened state (e.g., a partially-capped state) as shown in FIGS. 7A and 7B in which (i) the restraining force between the tulip 675 and the spanning member 674 is great enough to inhibit or even prevent axial movement of the spanning member 674 through the channel 877 when the balloon 630 is expanded in the disc space 607 and (ii) the restraining force between the spanning member 674 and the head 880 is low enough to at least partially permit polyaxial movement (e.g., rotation, pivoting) of the head 880 within the aperture 881, and thus polyaxial movement of the screw body 673 relative to the tulip 675, when the balloon 630 is expanded in the disc space 607. After expansion of the balloon 630, the nut 879 can be further tightened to a fully-tightened state (e.g., as described in detail above with reference to FIG. 3K) to increase the restraining force between the spanning member 674 and the head 880 to inhibit or even prevent polyaxial movement of the screw body 673 relative to the tulip 675 to maintain the created lordosis and spinal positioning. In some embodiments, the partially-tightened state can be between about 10-50%, about 10%, about 20%, about 30%, about 40%, and/or the like of the fully-tightened state. In at least some embodiments, for example, tightening (e.g., fully tightening) the nut 879 can include tightening the nut 879 to a maximum or fully-tightened torque value or range, and partially tightening the nut 879 includes tightening the nut 879 to a percentage (e.g., up to 10%, 20%, 30%, 40%, 50%, 60%, 70%, 80%, 90%, any percentage therebetween, and/or combinations thereof) of the maximum or fully-tightened torque value or range. Additionally, or alternatively, tightening (e.g., fully tightening) the nut 879 can include placing the nut 879 in contact with (e.g., threadably engaging) the surface 876 and rotating the nut 879 a predetermined number of degrees and/or rotations (e.g., full or partial rotations) relative to the surface 876, and partially tightening the nut 879 includes rotating the nut 879 a percentage (e.g., up to 10%, 20%, 30%, 40%, 50%, 60%, 70%, 80%, 90%, any percentage therebetween, and/or combinations thereof) of the predetermined number of degrees and/or rotations.

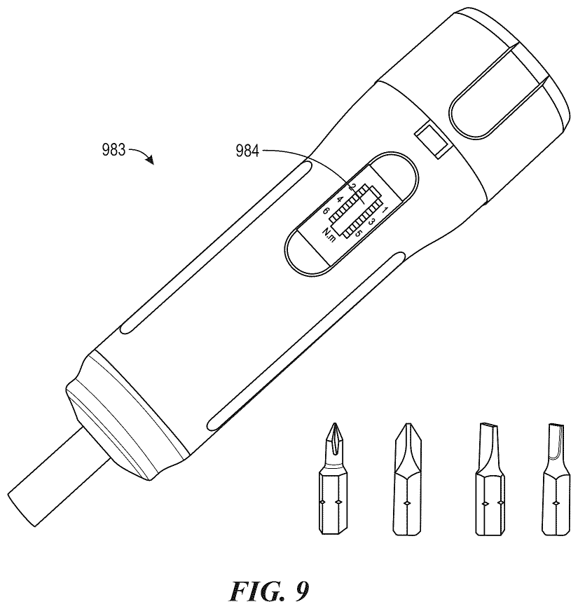

In some embodiments, the nut 879 can be tightened via a differential drive to achieve the differential tightening of the nut 879. That is, the differential driver can be configured to impart a first torque level for tightening the nut 879 to the partially-tightened state and a second torque level for tightening the nut 879 to the fully-tightened state. FIG. 9, for example, is a perspective view of a differential torque driver 983 in accordance with embodiments of the present technology. In the illustrated embodiment, the differential torque driver 983 includes an actuator 984 that can be actuated by a user (e.g., surgeon) to provide different levels of torque, such as the first and second torque levels. Additionally, or alternatively, the differential torque driver 983 can be configured to drive the nut 879 through a number of first rotations to tighten the nut 879 to the partially-tightened state and through a number of second rotations to tighten the nut 879 to the fully-tightened state.

### II. Selected Embodiments of Corridors for Accessing the L4/L5 Disc Space and/or the L5/S1 Disc Space

In many typical spinal surgical procedures, the L4/L5 disc space and the L5/S1 disc space cannot be accessed via a lateral approach. In particular, the iliac crest can block lateral access to the L4/L5 disc space using retractors and typical spinal instrumentation, and the iliac crest and the sacrum can block lateral access to the L5/S1 disc space using retractors and typical spinal instrumentation. Accordingly, these disc spaces are frequently accessed via an anterior approach that requires a vascular surgeon and poses increased risk to a patient. In some aspects of the present technology, the small and minimally invasive profile of the devices and systems of the preset technology described herein can allow direct lateral access to the L4/L5 and L5/S1 disc spaces via a lateral approach.

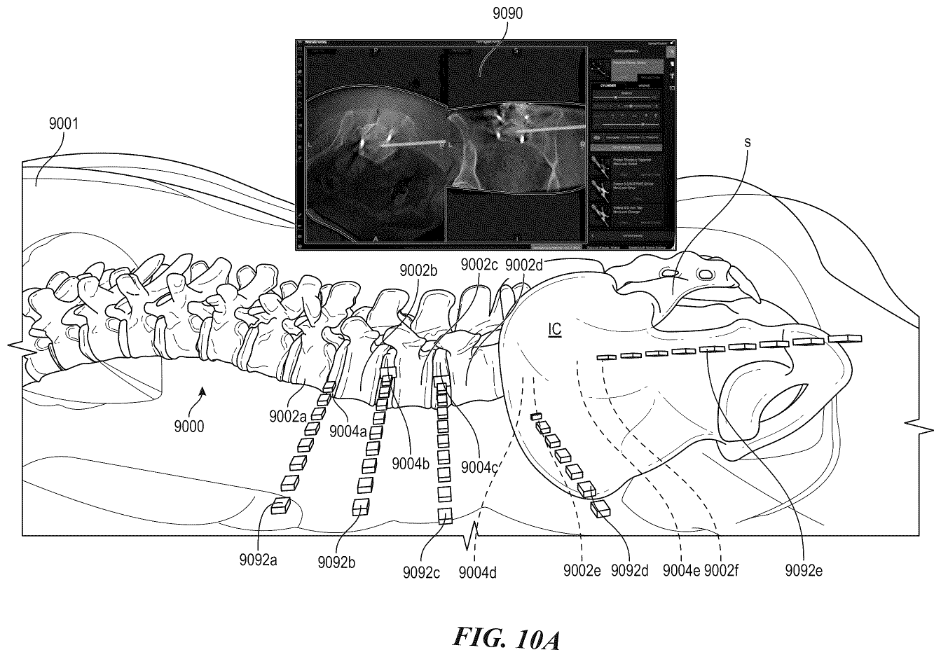

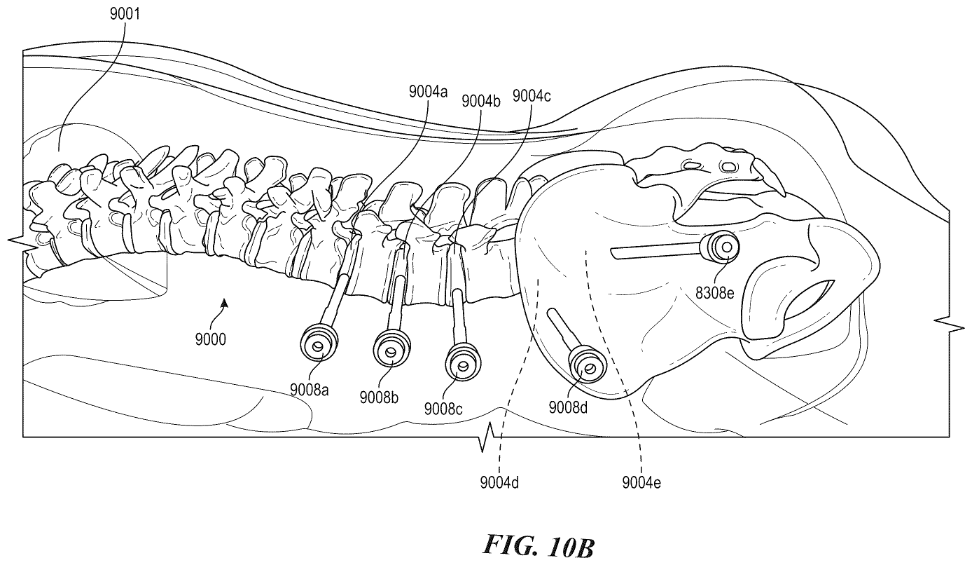

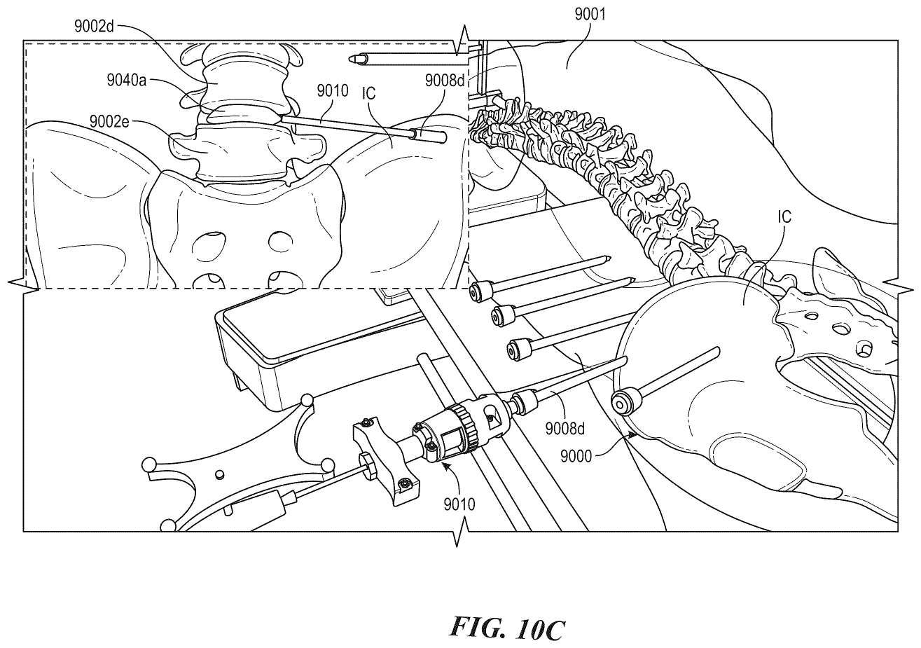

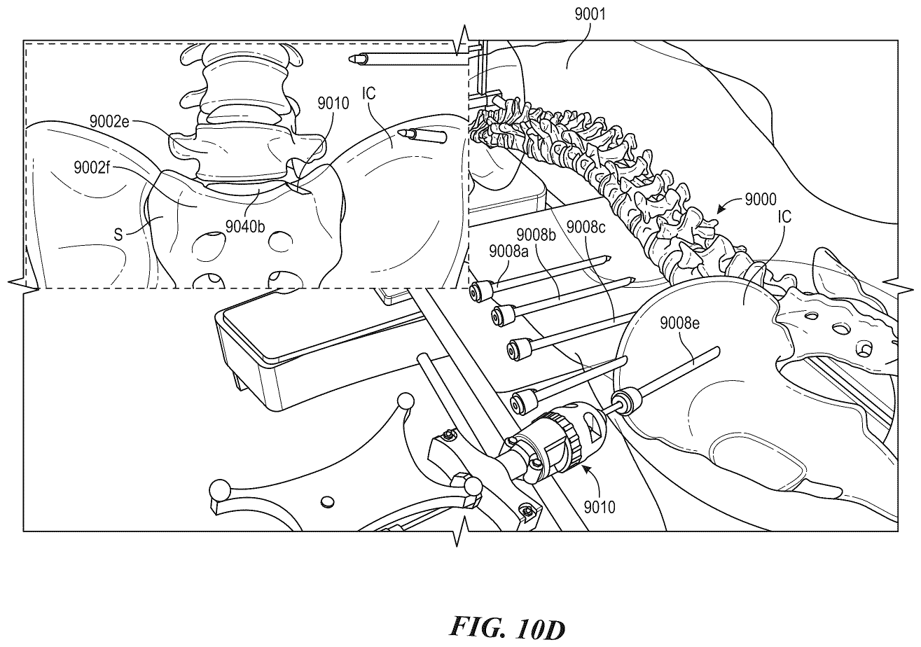

For example, FIGS. 10A-10D are different views of a spinal surgical procedure (e.g., a spinal surgical method) on a spine 9000 of a patient 9001 (shown as partially transparent for clarity) in accordance with additional embodiments of the present technology. More specifically, FIG. 90A is a side view (e.g., a lateral view) of a portion of the spine 9000 illustrating a navigation and trajectory planning step of the spinal surgical procedure in accordance with embodiments of the present technology. Referring to FIG. 10A, the spine 9000 includes a plurality of vertebrae 9002. More specifically, the vertebrae 9002 can include an L1 lumbar vertebra 9002a, an L2 lumbar vertebra 9002b, an L3 lumbar vertebra 9002c, an L4 lumbar vertebra 9002d, an L5 lumbar vertebra 9002e (obscured in FIG. 10A), and an S1 sacral vertebra 9002f (obscured in FIG. 10A). The vertebrae 9002 can be separated by corresponding discs 9004 (e.g., intervertebral discs; including individually identified first through fifth discs 9004a-e with the fourth and fifth discs 9004d-e obscured in FIG. 90A). A surgical navigation system 9090, such as the StealthStation Surgical Navigation System sold by Medtronic PLC, can be used to determine lateral trajectories 9092 to some or all of the discs 9004, such as first through fifth lateral trajectories 9092a-e to the first through fifth discs 9004a-e, respectively. In some aspects of the present technology, the fourth lateral trajectory 9092d to the fourth disc 9004d between the L4 lumbar vertebra 9002d and the L5 lumbar vertebra 9002e extends through an iliac crest IC of the patient 9001, and the fifth lateral trajectory 9092e to the fifth disc 8404e between the L5 lumbar vertebra 9002e and the S1 sacral vertebra 9002f extends through the iliac crest IC and a sacrum S of the spine 9000.

FIG. 10B is a side view (e.g., a lateral view) of a portion of the spine 9000 illustrating an access step of the spinal surgical procedure in accordance with embodiments of the present technology. One or more introducers 9008 can be inserted along one or more of the lateral trajectories 9092. For example, in the illustrated embodiment first through fifth introducers 9008a-e are inserted at least partially along the first through fifth lateral trajectories 9092a-e (FIG. 10A), respectively. The introducers 9008 can provide surgical access at least part way to discs 9004.

FIG. 10C is a side view (e.g., a lateral view) and an enlarged front view (e.g., anterior view) of a portion of the spine 9000 illustrating a first intervertebral device deployment step of the spinal surgical procedure in accordance with embodiments of the present technology. In the illustrated embodiment, a trocar 9010 can be inserted through the fourth introducer 9008d and used to access a disc space between the L4 lumbar vertebra 9002d and the L5 lumbar vertebra 9002e. A balloon can be deployed through the trocar 9010 to create lordosis and/or to distract the disc space, and/or a first intervertebral device 9040a can be deployed in the disc space through the trocar 9010 as described in detail herein. In some aspects of the present technology, the trocar 9010 can extend directly through the iliac crest IC of the patient 9001 due the small profile of the trocar 9010. The iliac crest IC can provide a rigid support for the trocar 9010 during operation, and the lateral path through the iliac crest IC reduces exposure to blood vessels, nerves, etc., as compared to, for example, an anterior approach.

FIG. 10D is a side view (e.g., a lateral view) and an enlarged front view (e.g., anterior view) of a portion of the spine 9000 illustrating a second intervertebral device deployment step of the spinal surgical procedure in accordance with embodiments of the present technology. In the illustrated embodiment, the same or a different trocar 9010 can be inserted through the fifth introducer 9008e and used to access a disc space between the L5 lumbar vertebra 9002e and the S1 sacral vertebra 9002f. A balloon can be deployed through the trocar 9010 to create lordosis and/or to distract the disc space, and/or a second intervertebral device 9040b can be deployed in the disc space through the trocar 9010 as described in detail herein. In some aspects of the present technology, the trocar 9010 can extend directly through the iliac crest IC the patient 9001 and the sacrum S of the spine 9000 due the small profile of the trocar 9010. The iliac crest IC and the sacrum S can provide a rigid support for the trocar 9010 during operation, and the lateral path through the iliac crest IC and the sacrum S reduces exposure to blood vessels, nerves, etc., as compared to, for example, an anterior approach. In some embodiments, the same or a different trocar 9010 can similarly be inserted through one or more of the first through third introducers 9008a-c and used to create lordosis, deploy intervertebral devices, etc., in the corresponding disc spaces accessed thereby.

### III. Selected Embodiments of Discectomy Devices, and Associated Systems and Methods

FIG. 11 is a perspective view of a distal portion of a discectomy device 1120 in accordance with embodiments of the present technology. The discectomy device 1120 can be inserted through a trocar and used to remove some or all of a diseased disc, such as described in detail above with reference to FIGS. 1C and 1D and blocks 282 and 283 of the method 280 of FIG. 2. Accordingly, the discectomy device 1120 can be utilized in the workflows of the spinal surgical procedures described in detail with reference to FIGS. 1A-2, FIGS. 3A-5, and/or elsewhere herein.

In the illustrated embodiment, the discectomy device 1120 includes an elongate shaft 1124 having a plurality of expandable bristles 1126 coupled to a distal end portion thereof. In some embodiments, the bristles 1126 are positioned in a helical orientation about the shaft 1124, such as extending between/coupled between adjacent helical windings of the shaft 1124. In other embodiments, the bristles 1126 can have other positions/orientations. During insertion of the discectomy device 1120 through a trocar or other introducer, the trocar can deflect the bristles 1126 toward the shaft 1124 to a delivery state. When inserted past a distal opening of the trocar, the bristles 1126 can self expand to an expanded state shown in FIG. 11. Accordingly, the bristles 1126 can have a relatively larger configuration/radius in the expanded state than in the delivery state. In some aspects of the present technology, this can enable the trocar to have a relatively small profile while still permitting the discectomy device 1120 to have a large footprint when expanded within the disc space for clearing of diseased disc material therein. The bristles 1126 can be formed from metal (e.g., nitinol) and, in the expanded state, the shaft 1124 can be translated proximally and distally relative to the trocar and/or rotated relative to the disc space and/or the trocar to mechanically engage and disrupt the disc material.

In some embodiments, the shaft 1124 can be shaped to deflect from a linear configuration when positioned outside the trocar. For example, in the illustrated embodiment the shaft 1124 includes a distal deflection section 1125. The distal deflection section 1125 can enable the discectomy device 1120 to be rotated/swept to a greater range of positions within the disc space to facilitate cleaning of some or all of the disc space.

### IV. Selected Embodiments of Balloon Devices, and Associated Systems and Methods

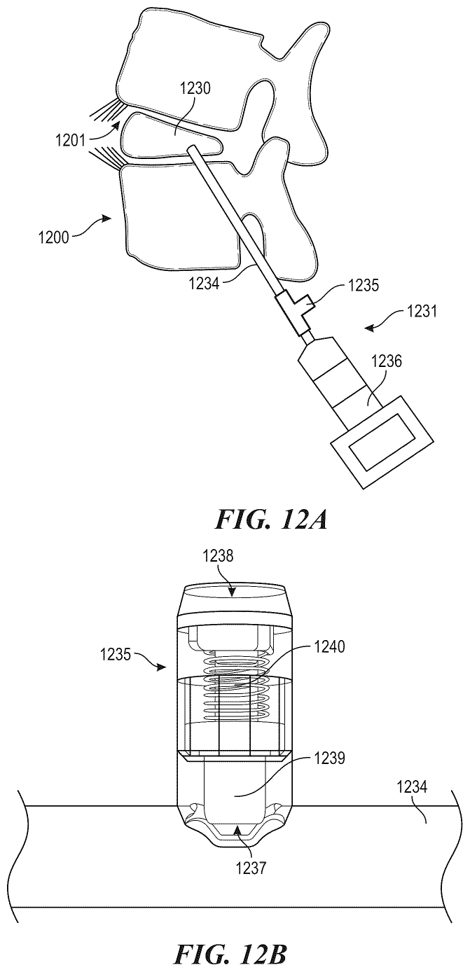

FIG. 12A is a side (e.g., lateral) view of a balloon device 1231 deployed and expanded within a disc space 1201 of a spine 1200 of a patient in accordance with embodiments of the present technology. The balloon device 1231 can be inserted through a trocar and expanded within the disc space 1201 to disrupt disc material within the disc space 1201 and/or to enlarge the disc space 1201 (e.g., by lifting a vertebra adjacent to the disc space 1201), such as described in detail above with reference to FIGS. 1E-1H and blocks 284 and 285 of the method 280 of FIG. 2 and FIGS. 3E-3K and blocks 584, 585, and 587 of the method 580 of FIG. 5. Accordingly, the balloon device 1231 can be utilized in the workflows of the spinal surgical procedures described in detail with reference to FIGS. 1A-2, FIGS. 3A-5, and/or elsewhere herein.

In the illustrated embodiment, the balloon device 1231 includes a balloon 1230 operably coupled to an inflation assembly 1236. In some embodiments, an inflation shaft 1234 (e.g., the outer balloon shaft 134 of FIGS. 1F and 1H) fluidly connects the balloon 1230 to the inflation assembly 1236. The inflation assembly 1236 is operable to drive an inflation fluid (e.g., air, liquid, contrast liquid, saline, and/or mixtures thereof) through the inflation shaft 1234 to inflate the balloon 1230. In the illustrated embodiment, a pressure-relief valve 1235 is positioned along the inflation shaft 1234. The pressure-relief valve 1235 can be positioned along the inflation shaft 1234 outside a patient (e.g., not within the body of the patient) and configured to open and vent at least a portion of the inflation fluid when a pressure in the inflation shaft 1234 and/or the balloon 1230 exceeds a predetermined level. In some embodiments, the pressure-relief valve 1235 is a passive valve configured to automatically open when the pressure exceeds the predetermined level.

For example, FIG. 12B is a partially-transparent side view of the pressure-relief valve 1235 coupled to a portion of the inflation shaft 1234 in accordance with embodiments of the present technology. In the illustrated embodiment, the pressure-relief valve 1235 defines a fluid path therethrough from (i) a first opening 1237 that opens to the inflation shaft 1234 to (ii) a second opening 1238 that opens to the atmosphere or other source. The pressure-relief valve 1235 can further include a plunger 1239 coupled to a biasing member 1240 between the first and second openings 1237, 1238. The biasing member 1240 can be a compression spring or other type of spring having a spring force tuned to a desired pressure-responsiveness of the pressure-relief valve 1235. That is, the biasing member 1240 can operate to close the fluid path between the first and second openings 1237, 1238 when the pressure within the inflation shaft 1234 is below the preselected level (e.g., as shown in FIG. 12B), and open the fluid path between the first and second openings 1237, 1238 when the pressure within the inflation shaft 1234 exceeds the preselected level. More specifically, the biasing member 1240 can bias the plunger 1239 toward/into the first opening 1237 to seal the first opening 1237 and inhibit or even prevent fluid flow between the first and second openings 1237, 1238 when the pressure is below the preselected level. When the pressure exceeds the preselected level, the pressure in the inflation shaft 1234 can drive plunger 1239 upward against the biasing force of the biasing member 1240 to move the plunger 1239 away from/out of the first opening 1237 to unseal the first opening 1237 and permit fluid flow between the first and second openings 1237, 1238.

Referring to FIGS. 12A and 12B, in some aspects of the present technology the pressure-relief valve 1235 can inhibit or even prevent over pressurization of the balloon 1230 that might cause the balloon 1230 to rupture. When the inflation fluid comprises a contrast agent, rupture of the balloon 1230 can lead to complications/difficulties in visualizing downstream steps of a spinal surgical procedure via radiographic and/or other imaging techniques.

### V. Selected Embodiments of Intervertebral Devices, and Associated Systems and Methods

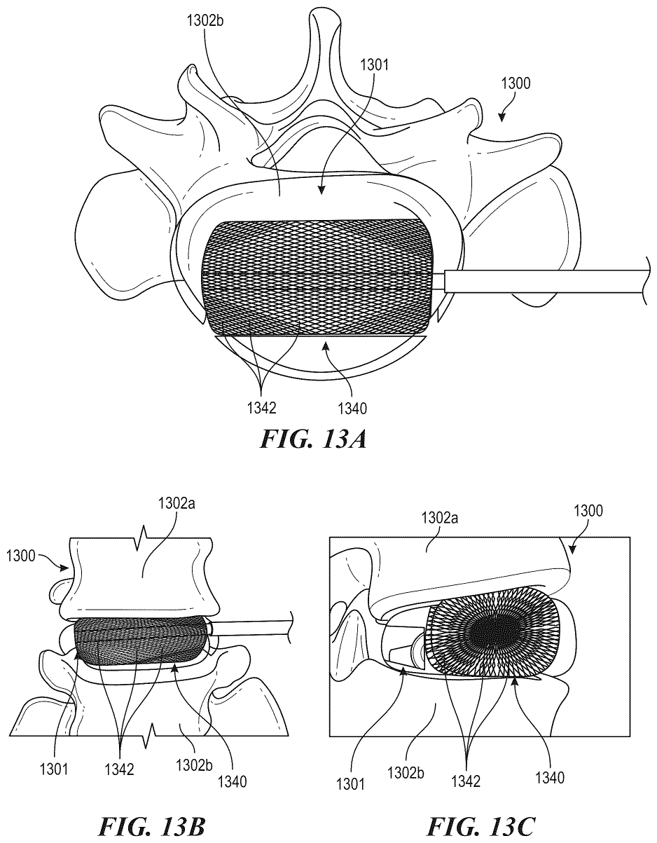

FIGS. 13A-14C illustrate embodiments of intervertebral devices that can be inserted through a trocar and deployed within a disc space, such as described in detail above with reference to FIGS. 1I-1K and blocks 286 and 287 of the method 280 of FIG. 2, and FIGS. 3L and 3M and blocks 589 and 590 of the method 580 of FIG. 5. Accordingly, the embodiments described with reference to FIGS. 13A-14C can be utilized in the workflows of the spinal surgical procedures described in detail with reference to FIGS. 1A-2, FIGS. 3A-5, and/or elsewhere herein.

FIGS. 13A-13C are a top (e.g., axial) view, a back (e.g., posterior) view, and a side (e.g., lateral) view, respectively, of an intervertebral device 1340 deployed and expanded within a disc space 1301 of a spine 1300 of a patient in accordance with embodiments of the present technology. Referring to FIGS. 13A-13C, the intervertebral device 1340 can comprise a braid of filaments 1342. In the illustrated embodiment, the intervertebral device 1340 is configured to expand to generally rectilinear shape (e.g., a somewhat rectangular prism). In some aspects of the present technology, this shape can help distribute forces evenly between the intervertebral device 1340 and an upper vertebra 1302a (not shown in FIG. 13A) and a lower vertebra 1302b adjacent the disc space 1301 to, for example, inhibit or even prevent subsidence of the intervertebral device 1340 after deployment.

FIG. 14A is a perspective view of an intervertebral device 1440 in accordance with embodiments of the present technology. FIG. 14B is an enlarged perspective view of a portion of the intervertebral device 1440 of FIG. 14A in accordance with embodiments of the present technology. Referring to FIGS. 14A and 14B, the intervertebral device 1440 can comprise a braid of filaments 1442 that is filled with a fill material 1460. In the illustrated embodiment, the fill material 1460 comprises a plurality of particles 1462 (FIG. 14B) that, when filled within an intervertebral device, can form a gabion structure. The particles 1462 can be formed from trabecular bone tissue, titanium, metal, silica, metal, biomaterial, sand, demineralized bone, and/or the like. The particles 1462 can have a three-dimensional lattice structure with an irregular (e.g., roughened) outer surface. The irregular outer surfaces can provide a high-friction interface and/or interlock interface between the particles 1462 when filled with the intervertebral device—for example, providing a “Velcro-like” coupling between the particles 1462 to enhance a gabion effect. In some embodiments, the particles 1462 can each comprise a unitary body comprising a pair of rectangular prisms extending orthogonal to and intersecting one another. The shape of the particles 1462 can cause the fill material 1460 to form a strong gabion structure in which a substantial portion (e.g., 90% or more) of an axial force (e.g., in the direction of an arrow A in FIG. 14A) is transmitted axially rather than radially.

In some embodiments, when deployed in a disc space between adjacent vertebrae, the intervertebral device 1440 can be configured to substantially maintain a height H (FIG. 14A) thereof even when the intervertebral device 1440 is subject to substantial loads, such as in the axial direction indicated by the arrow A. This can inhibit or even prevent subsidence of the intervertebral device 1440 into the adjacent vertebrae. For example, FIG. 14C is a graph illustrating the height H of the intervertebral device 1440 in millimeters along the Y-axis versus a loading force in pounds along the X-axis in accordance with embodiments of the present technology. In the illustrated embodiment, as shown by the curve 1464, the intervertebral device 1440 can inhibit relatively even and low reduction in the height H over a large range of loading forces. Curves 1465 represent the same subsidence/loss of height testing for other intervertebral devices (e.g., the FlareHawk7 expandable lumbar interbody feature manufactured by Accelus, the MOD-EX PL interbody system manufactured by NuVasive, the OptiMesh implant manufactured by Spineology, the RISE-L expandable lumbar interbody spacer manufactured by Globus Medical, titanium alloy implants used in anterior lumbar interbody fusion (ALIF) surgery, etc.), the that show greater height loss and subsidence over the same range of loading forces.

Referring to FIGS. 14A and 14B, the filaments 1442 can be formed of a metal such as titanium (Ti), cobalt chromium (CoCr), Ti/CoCr, and/or the like, and may be coated with one or more other materials (e.g., drug-eluting materials). In some aspects of the present technology, TI/CoCr advantageously provides for strong and rapid osseointegration. The fill material 1460 can be formed of polyetheretherketone (PEEK), hydroxyapatite (HA), tricalcium phosphate (TCP), biphasic calcium phosphate (BCP; a combination of HA and TCP), and/or other materials that provide advantages such as, for example, bioactivity, osseointegration, biocompatibility, and non-cytotoxicity. The fill material 1460 can be porous to allow for bone to grow throughout the pores to completely lock the gabion structure of the particles 1462 and to conduct continuous osteoconduction through the pores. The incorporation of HA, TCP, and/or other osseoinductive agents into the fill material 1460 can promote faster bone growth through the pores of the fill material 1460. In some embodiments, the fill material 1460 can comprise a radiopaque material (e.g., HA). In some embodiments, the particles 1462 are formed by extruding or molding a compound material including radiopaque and non-radiopaque materials. For example, the compound material can comprise PEEK (non-radiopaque) and HA (radiopaque). Often, during such an extrusion or molding process, the PEEK will form a surface film that encapsulates the HA and/or other radiopaque materials—inhibiting or even preventing radiopacity of the particles 1462. However, in some embodiments the extruded or molded material is cut to form the individual particles 1462 such that the radiopaque material is exposed at two ends of each of the particles 1462—allowing for radiopacity of the particles 1462.

In some embodiments, an elasticity and/or modulus of the intervertebral device 1440 can be customized (e.g., for an individual patient) in several ways such that, for example, the intervertebral device 1440 has a desired overall elasticity and/or modulus that can be similar to that of vertebral bone of the patient. For example, the material of the fill material 1460 can be changed to include more, less, or different of the materials described herein. Likewise, a manufacturing process used to form the fill material 1460 can be adjusted by changing, for example, melting parameters, injection/extrusion parameters, cooling parameters, annealing parameters, and/or the like. Additionally, the geometry of the particles 1462 can be modified. For example, if the particles 1462 are made to have rounder edges, the edges can be more compliant and/or provide more degrees of freedom to the gabion structure of the fill material 1460 (e.g., increasing the elasticity and/or the modulus of the intervertebral device 1440). Likewise, thinner edges of the particles 1462 allow for more bulk flex in the gabion structure of the fill material 1460 (e.g., increasing the elasticity and/or the modulus of the intervertebral device 1440), while more compliant, thicker edges of the particles 1462 allow for less flex in the gabion structure of the fill material 1460 (e.g., decreasing the elasticity and/or the modulus of the intervertebral device 1440). For example, the particles 1462 can have any of the shapes described in detail in U.S. patent application Ser. No. 18/619,125, filed Mar. 27, 2024, and titled “INTERVERTEBRAL DEVICES, AND ASSOCIATED SYSTEMS AND METHODS,” which is incorporated by reference herein in its entirety (e.g., in particular, as described in detail with reference to FIGS. 40A-48B thereof).

Likewise, mixing different types of the particles 1462 (e.g., having different materials, shapes, sizes, etc.) can create differing characteristics of the intervertebral device 1440. For example, the particles 1462 can comprise a mixture of larger particles (e.g., gabion particles; e.g., formed of PEEK mixed with BCP) and smaller particles (e.g., formed of demineralized bone (DBM)). The larger particles can be configured to form the gabion structure of the fill material 1460 while the smaller particles can slightly suspend the larger particles to allow for greater compliance when the intervertebral device 1440 is initially deployed, until the larger particles are compressed to the point of contact and interlock. In some aspects of the present technology, this can allow the intervertebral device 1440 to be more moldable to the surface topography of the adjacent vertebrae before locking static. Moreover, the smaller particles can fill the pores of the larger particles to further restrict degrees of freedom in the fill material 1460 and stiffen the intervertebral device 1440.

In some embodiments, the elasticity and/or modulus of the fill material 1460 can be selected to match or generally match (e.g., be slightly below) a corresponding elasticity and/or modulus of the bone (e.g., vertebral bone) of a specific patient. For example, prior to implantation of the intervertebral device 1440, the patient can undergo preoperative imaging (e.g., x-ray imaging) to determine a density, elasticity, and/or modulus of the bone of the patient. The fill material 1460 can then be selected to match or generally match the density, elasticity, and/or modulus of the bone by varying any of the factors described in detail above.

In some embodiments, the intervertebral device 1440 is configured to be drug-eluting. More specifically, the filaments 1442, the fill material 1460, and/or other components of the intervertebral device 1440 can be coated with a material configured to dissolve after implantation of the intervertebral device 1440 and/or be absorbed by anatomy of the patient surrounding the intervertebral device 1440. The material can be selected to inhibit osteogenesis, promote osteogenesis, and/or the like.

### VI. Selected Embodiments of Retention Devices, and Associated Systems and Methods

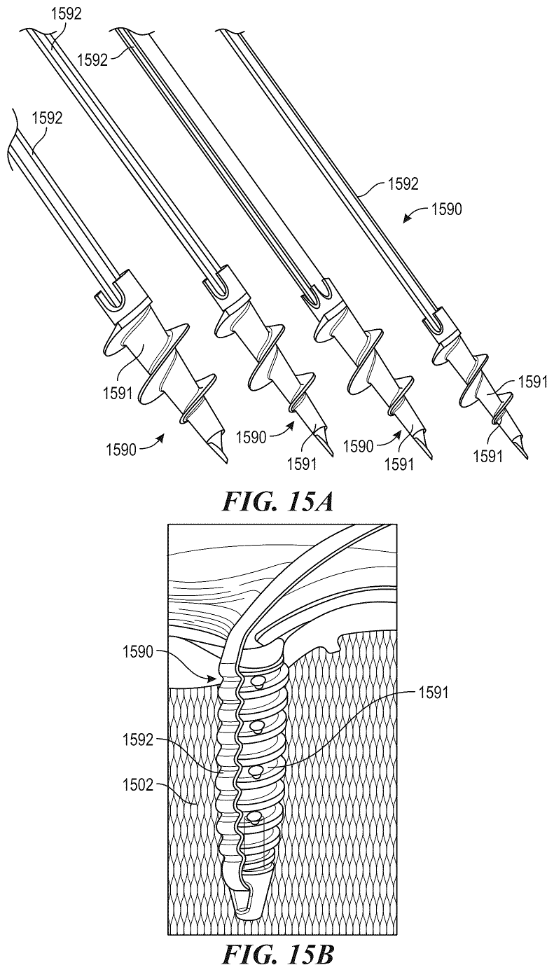

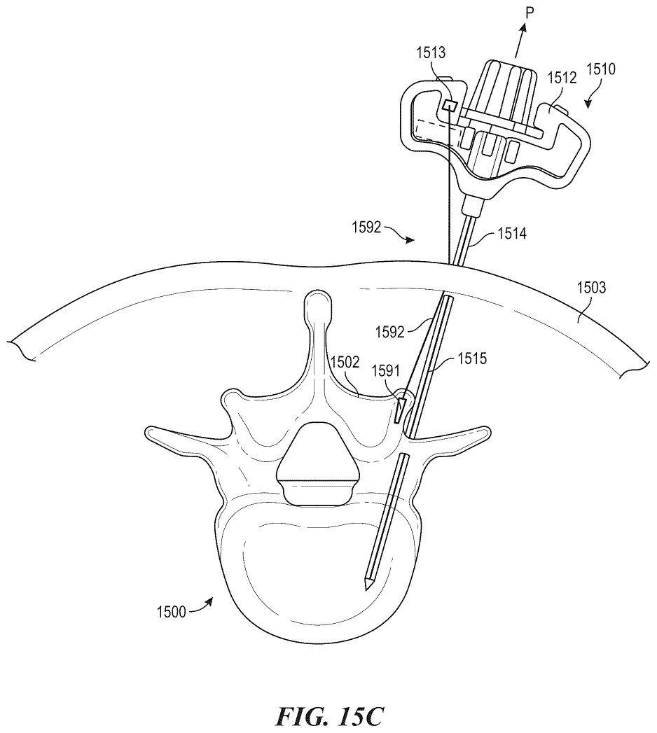

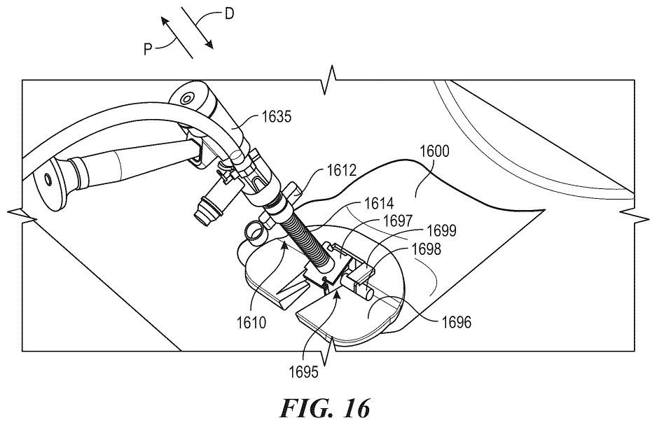

FIGS. 15A-16 illustrate embodiments of retention devices that can be used to retain a trocar in position relative to a patient (e.g., a disc space of a patient). The various retention devices can be used to secure and retain a trocar and/or other access device during any of the steps of the various workflows of the spinal surgical procedures described in detail with reference to FIGS. 1A-2, FIGS. 3A-5, and/or elsewhere herein.

FIG. 15A illustrates several (e.g., four) suture anchors 1590 in accordance with embodiments of the present technology. Likewise, FIG. 15B illustrates another suture anchor 1590 secured within bone (e.g., tissue) 1502 in accordance with embodiments of the present technology. In the illustrated embodiments, each of the suture anchors 1590 includes an anchor 1591 coupled to one or more sutures 1592. In some embodiments, the anchors 1591 are threaded or otherwise shaped to be able to be driven (e.g., rotated) into and retained/secured within bone of the patient. For example, referring to FIG. 15B, the anchor 1591 can be inserted through a trocar or other access device (not shown) and driven into the vertebra 1502 and secured therein. The suture 1592 can extend from the anchor and at least partially out of the patient and can be secured to the trocar to inhibit or even prevent movement of the trocar in a direction away from the patient.

FIG. 15C, for example, is a top (e.g., axial) view of a trocar 1510 for providing access (e.g., lateral, transpedicular, transfacet, transforaminal, trans-iliac, trans-sacral, and/or other access) to a vertebra or disc of a spine 1500 of a patient in accordance with embodiments of the present technology. The trocar 1510 includes a handle 1512 coupled to a hollow cannula 1514. In the illustrated embodiment, the trocar 1510 is secured to one of the suture anchors 1590 of FIGS. 15A and 15B. More specifically, the anchor 1591 can be positioned and secured within vertebral bone of a vertebra 1502 of the spine 1500, and the suture 1592 can extend from the anchor 1591, out of skin 1503 of the patient, and to the handle 1512. In some embodiments, the handle 1512 includes a retaining feature 1513, such as a hook, clamp, and/or the like, for securing a proximal end portion of the suture 1592. The suture 1592 can be brought into tension such that the suture anchor 1590 inhibits or even prevents movement of the trocar 1510 in a direction indicated by an arrow P away from the spine 1500 and the skin 1503 of the patient.

The suture anchor 1590 can be deployed through the cannula 1514. For example, during initial positioning of the trocar 1510, the cannula 1514 can be steered toward a suitable anchoring site within the vertebra 1502 or elsewhere, and the anchor 1591 can be advanced through the cannula 1514 and driven into the anchor site (e.g., via a driver extending through the cannula 1514 and configured to rotate the anchor 1591 into the vertebra 1502). During deployment, the suture 1592 can extend through the cannula 1514 and be accessible at the handle 1512. Once the anchor 1591 is secured, the suture 1592 can be removed from the cannula 1514 and secured to the retaining feature 1513 outside of the cannula 1514 such that other instruments (e.g., discectomy devices, balloons, intervertebral devices, etc.) can be advanced through the cannula 1514. For example, in the illustrated embodiment, the cannula 1514 includes a slit 1515 extending longitudinally there along and through which the suture 1592 can be passed after deployment of the anchor 1591. In some embodiments, multiple ones of the suture anchors 1590 can be used to secure the trocar 1510 to the patient. In some embodiments, to remove the trocar 1510 from the patient, the suture 1592 can be cut or otherwise removed from the retaining feature 1513 to allow for movement of the trocar 1510 in the direction of the arrow P away from the patient. The anchor 1591 can be removed via the trocar 1510, or left within the patient.

FIG. 16 is a perspective view of a trocar 1610 for providing access to a vertebra or disc of a patient 1600 in accordance with embodiments of the present technology. The trocar 1610 includes a handle 1612 coupled to a hollow cannula 1614. A balloon device 1635 is shown inserted through the trocar 1610. In the illustrated embodiment, the trocar 1610 includes a retaining feature 1695 configured to releasably engage/mate with a retaining plate 1696 positioned against the patient 1600 (e.g., skin of the patient). More specifically, the retaining feature 1695 can include a hub 1697 positioned at least partially around the cannula 1614, and one or more flanges 1698 extending from the hub 1697 (e.g., a pair of the flanges 1698 extending from opposite sides of the hub 1697). The retaining plate 1696 can include a flange or rib 1699 configured to receive the one or more flanges 1698 therein/thereunder. The retaining feature 1695 is shown engaged with the retaining plate 1696 in a retained position in FIG. 16 with the one or more flanges 1698 positioned within the rib 1699. In the retained position, the engagement of the retaining feature 1695 with the retaining plate 1696 inhibits or even prevents movement of the trocar 1610 in the direction of an arrow D in a direction toward the patient 1600 and in the direction of an arrow P in a direction away from the patient 1600. In some embodiments, the retaining feature 1695 (e.g., the hub 1697) can be slid along the cannula 1614 and subsequently secured/retained/locked thereto to facilitate retaining of the trocar 1610 at a selected insertion depth.

Referring to FIGS. 10C and 10D, in some embodiments one or both of the trocars 9010 used to access the L4/L5 disc space and/or the L5/S1 disc space can include a retaining/anchoring feature for anchoring to the iliac crest IC and/or the sacrum S. For example, an outer surface of the trocar 9010 can be ribbed, threaded, and/or the like to provide anchoring within the iliac crest IC and/or the sacrum that inhibits or even prevents the trocar 9010 from being inadvertently pushed into and/or withdrawn out of the iliac crest IC and/or the sacrum S.

### VII. EXAMPLES

The following examples are illustrative of several embodiments of the present technology:

1. A method of treating a spine of a patient, the method comprising:
coupling a fixation member to a first vertebra of the spine;
positioning a portion of a spanning member in a head of the fixation member;
partially tightening a nut of the fixation member against the portion of the spanning member;
causing the first vertebra to rotate relative to a second vertebra of the spine to create lordosis between the first vertebra and the second vertebra; and
fully tightening the nut against the portion of the spanning member to maintain the created lordosis.

2. The method of example 1 wherein:
partially tightening the nut includes applying a first amount of torque to the nut, and
fully tightening the nut includes applying a second amount of torque to the nut, the second amount greater than the first amount.

3. The method of example 2 wherein the first amount of torque is up to 50% of the second amount of torque.
4. The method of any of examples 1-3 wherein:
partially tightening the nut includes rotating the nut relative to the head through a first number of rotations, and
fully tightening the nut includes rotating the nut relative to the head through a second number of rotations, wherein the second number of rotations is less than the first number of rotations.

5. The method of example 4 wherein the first number of rotations is up to 80% of a sum of the first number of rotations and the second number of rotations.

6. The method of any of examples 1-5 wherein partially tightening the nut includes:
allowing the first vertebra and/or the second vertebra to move relative to the head, and
inhibiting the head from moving relative to the spanning member.

7. The method of any of examples 1-6 wherein fully tightening the nut includes:
inhibiting the first vertebra and/or the second vertebra from moving relative to the head, and
inhibiting the head from moving relative to the spanning member.

8. The method of any of examples 1-7 wherein:
coupling the fixation member to the first vertebra includes positioning a screw body of the fixation member at least partially within the first vertebra, wherein the screw body is rotatably coupled to the head; and
partially tightening the nut includes allowing the screw body to rotate relative to the head while inhibiting the head from moving longitudinally along the spanning member.

9. The method of any of examples 1-8 wherein the fixation member is a first fixation member, the head is a first head of the first fixation member, the portion is a first portion of the spanning member, the nut is a first nut of the first fixation member, and the method further comprises:
coupling a second fixation member to the second vertebra;
positioning a second portion of the spanning member in a second head of the second fixation member;
partially tightening a second nut of the second fixation member against the second portion; and
after creating the lordosis between the first vertebra and the second vertebra, fully tightening the second nut against the second portion of the spanning member.

10. The method of any of examples 1-9 wherein causing the first vertebra to rotate relative to the second vertebra includes:
positioning a device in a disc space at least partially between the first vertebra and the second vertebra, and
causing the device to expand.

11. A system for treating a spine of a patient, the system comprising:
a trocar configured to provide access to a diseased disc within a disc space of the spine;
an anchoring device configured to restrict movement of the trocar in at least one direction relative to the disc space;
a discectomy device insertable through the trocar to disrupt at least a portion of the diseased disc;
an intervertebral device insertable through the trocar into the disc space and configured to expand within the disc space; and
a fill material configured to fill the intervertebral device.

12. The system of example 11 wherein the anchoring device comprises an anchor configured to be driven into bone adjacent the disc space and a suture coupled to the anchor and configured to be coupled to a portion of the trocar to inhibit a range of proximally-directed movement of the trocar relative to the disc space.

13. The system of example 12 wherein the trocar includes a cannula having a slit extending longitudinally there along, wherein the slit is configured to allow the suture to be removed from within the cannula after the anchor is driven into the bone.

14. The system of any of examples 11-13 wherein the trocar includes a retaining feature and wherein the anchoring device comprises a retaining plate configured to be positioned against the patient and releasably couple the retaining feature to inhibit a range of proximally-and/or distally-directed movements of the trocar relative to the disc space. 15. The system of example 14 wherein the retaining feature includes a hub positioned at least partially around a cannula of the trocar and a flange extending from the hub, and wherein the retaining plate includes a rib that defines a recessed area configured to receive the flange. 16. A method of treating a spine of a patient, the method comprising:
inserting a trocar to proximate a diseased disc within a disc space of the spine;
anchoring the trocar relative to the disc space to restrict movement of the trocar in at least one direction relative to the disc space;
inserting a discectomy device through the trocar;
disrupting at least a portion of the diseased disc with the discectomy device;
inserting an intervertebral device through the trocar into the disc space;
expanding the intervertebral device within the disc space; and
filling the intervertebral device with a fill material.

17. The method of example 16 wherein anchoring the trocar relative to the disc space includes:
positioning the trocar proximate an anchoring site within a vertebra of the spine;
advancing an anchor through the trocar to proximate the anchoring site;
driving the anchor into the anchoring site;
removing a suture from the trocar, the suture coupled to the anchor; and
securing the suture to a portion of the trocar to inhibit a range of proximally-directed movement of the trocar relative to the disc space.

18. The method of example 17 wherein the trocar includes a cannula having a slit extending longitudinally there along, and wherein removing the suture from the trocar includes removing at least a portion of the suture from within the cannula through the slit.

19. The method of any of examples 16-18 wherein:
the trocar includes a retaining feature; and
anchoring the trocar includes—
   positioning
   a retaining plate against the patient, and coupling the retaining feature to the retaining plate to inhibit a range of proximally- and/or distally-directed movements of the trocar relative to the disc space.

20. The method of example 19 wherein:
the retaining feature includes a hub positioned at least partially around a cannula of the trocar and a flange extending from the hub;
the retaining plate includes a rib that defines a recessed area configured to receive the flange; and
coupling the retaining feature to the flange includes positioning the rib at least partially within the recessed area.

### VIII. Conclusion

All numeric values are herein assumed to be modified by the term about whether or not explicitly indicated. The term about, in the context of numeric values, generally refers to a range of numbers that one of skill in the art would consider equivalent to the recited value (e.g., having the same function and/or result). For example, the term about can refer to the stated value plus or minus ten percent. For example, the use of the term about 100 can refer to a range of from 90 to 110, inclusive. In instances in which the context requires otherwise and/or relative terminology is used in reference to something that does not include, or is not related to, a numerical value, the terms are given their ordinary meaning to one skilled in the art.

The above detailed description of embodiments of the present technology are not intended to be exhaustive or to limit the technology to the precise forms disclosed above. Although specific embodiments of, and examples for, the technology are described above for illustrative purposes, various equivalent modifications are possible within the scope of the technology, as those skilled in the relevant art will recognize. For example, although steps may be presented in a given order, in other embodiments, the steps may be performed in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

As used herein, the phrase and/or as in A and/or B refers to A alone, B alone, and A and B. Additionally, the term comprising is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A method of treating a spine of a patient, the method comprising:
coupling a fixation member to a first vertebra of the spine;
positioning a portion of a spanning member in a head of the fixation member;
partially tightening a nut of the fixation member against the portion of the spanning member;
causing the first vertebra to rotate relative to a second vertebra of the spine to create lordosis between the first vertebra and the second vertebra; and
fully tightening the nut against the portion of the spanning member to maintain the created lordosis.

2. The method of claim 1 wherein:
partially tightening the nut includes applying a first amount of torque to the nut, and
fully tightening the nut includes applying a second amount of torque to the nut, the second amount greater than the first amount.

3. The method of claim 2 wherein the first amount of torque is up to 50% of the second amount of torque.

4. The method of claim 1 wherein:
partially tightening the nut includes rotating the nut relative to the head through a first number of rotations, and
fully tightening the nut includes rotating the nut relative to the head through a second number of rotations, wherein the second number of rotations is less than the first number of rotations.

5. The method of claim 4 wherein the first number of rotations is up to 80% of a sum of the first number of rotations and the second number of rotations.

6. The method of claim 1 wherein partially tightening the nut includes:
allowing the first vertebra and/or the second vertebra to move relative to the head, and
inhibiting the head from moving relative to the spanning member.

7. The method of claim 1 wherein fully tightening the nut includes:
inhibiting the first vertebra and/or the second vertebra from moving relative to the head, and
inhibiting the head from moving relative to the spanning member.

8. The method of claim 1 wherein:
coupling the fixation member to the first vertebra includes positioning a screw body of the fixation member at least partially within the first vertebra, wherein the screw body is rotatably coupled to the head; and
partially tightening the nut includes allowing the screw body to rotate relative to the head while inhibiting the head from moving longitudinally along the spanning member.

9. The method of claim 1 wherein the fixation member is a first fixation member, the head is a first head of the first fixation member, the portion is a first portion of the spanning member, the nut is a first nut of the first fixation member, and the method further comprises:
coupling a second fixation member to the second vertebra;
positioning a second portion of the spanning member in a second head of the second fixation member;
partially tightening a second nut of the second fixation member against the second portion; and
after creating the lordosis between the first vertebra and the second vertebra, fully tightening the second nut against the second portion of the spanning member.

10. The method of claim 1 wherein causing the first vertebra to rotate relative to the second vertebra includes:
positioning a device in a disc space at least partially between the first vertebra and the second vertebra, and
causing the device to expand.

11. A system for treating a spine of a patient, the system comprising:
a trocar configured to provide access to a diseased disc within a disc space of the spine;
an anchoring device configured to restrict movement of the trocar in at least one direction relative to the disc space;
a discectomy device insertable through the trocar to disrupt at least a portion of the diseased disc;
an intervertebral device insertable through the trocar into the disc space and configured to expand within the disc space; and
a fill material configured to fill the intervertebral device.

12. The system of claim 11 wherein the anchoring device comprises an anchor configured to be driven into bone adjacent the disc space and a suture coupled to the anchor and configured to be coupled to a portion of the trocar to inhibit a range of proximally-directed movement of the trocar relative to the disc space.

13. The system of claim 12 wherein the trocar includes a cannula having a slit extending longitudinally there along, wherein the slit is configured to allow the suture to be removed from within the cannula after the anchor is driven into the bone.

14. The system of claim 11 wherein the trocar includes a retaining feature and wherein the anchoring device comprises a retaining plate configured to be positioned against the patient and releasably couple the retaining feature to inhibit a range of proximally-and/or distally-directed movements of the trocar relative to the disc space.

15. The system of claim 14 wherein the retaining feature includes a hub positioned at least partially around a cannula of the trocar and a flange extending from the hub, and wherein the retaining plate includes a rib that defines a recessed area configured to receive the flange.

16. A method of treating a spine of a patient, the method comprising:
inserting a trocar to proximate a diseased disc within a disc space of the spine;
anchoring the trocar relative to the disc space to restrict movement of the trocar in at least one direction relative to the disc space;
inserting a discectomy device through the trocar;
disrupting at least a portion of the diseased disc with the discectomy device;
inserting an intervertebral device through the trocar into the disc space;
expanding the intervertebral device within the disc space; and
filling the intervertebral device with a fill material.

17. The method of claim 16 wherein anchoring the trocar relative to the disc space includes:
positioning the trocar proximate an anchoring site within a vertebra of the spine;
advancing an anchor through the trocar to proximate the anchoring site;
driving the anchor into the anchoring site;
removing a suture from the trocar, the suture coupled to the anchor; and
securing the suture to a portion of the trocar to inhibit a range of proximally-directed movement of the trocar relative to the disc space.

18. The method of claim 17 wherein the trocar includes a cannula having a slit extending longitudinally there along, and wherein removing the suture from the trocar includes removing at least a portion of the suture from within the cannula through the slit.

19. The method of claim 16 wherein:
the trocar includes a retaining feature; and
anchoring the trocar includes—
positioning a retaining plate against the patient, and
coupling the retaining feature to the retaining plate to inhibit a range of proximally- and/or distally-directed movements of the trocar relative to the disc space.

20. The method of claim 19 wherein:
the retaining feature includes a hub positioned at least partially around a cannula of the trocar and a flange extending from the hub;
the retaining plate includes a rib that defines a recessed area configured to receive the flange; and
coupling the retaining feature to the flange includes positioning the rib at least partially within the recessed area.
